(19) United States
(12) Patent Application Publication
(10) Pub. No.: US 2015/0233835 A1
(43) Pub. Date: Aug. 20, 2015

(54) ANALYSIS APPARATUS AND ELECTRONIC DEVICE

(71) Applicant: Seiko Epson Corporation, Tokyo (JP)

(72) Inventors: Mamoru SUGIMOTO, Chino (JP); Megumi ENARI, Suwa (JP)

(21) Appl. No.: 14/621,913

(22) Filed: Feb. 13, 2015

(30) Foreign Application Priority Data
Feb. 17, 2014 (JP) 2014-027822

(51) Int. Cl. G01N 21/65; A61B 5/00; G06F 19/00

(52) U.S. Cl. CPC G01N 21/658; G06F 19/3431; A61B 5/0075; A61B 5/7278; A61B 5/742

(57) ABSTRACT

An analysis apparatus includes an electric field enhancing element including a metallic layer, a light-transmissive layer, and a plurality of metallic particles arranged in a first direction and a second direction intersecting with the first direction; a light source irradiating the electric field enhancing element with at least one of linearly polarized light polarized in the first direction, linearly polarized light polarized in the second direction, and circularly polarized light; and a detector, in which localized surface plasmon and propagating surface plasmon are electromagnetically interacted, and when a thickness of the light-transmissive layer is G [nm], an effective reflective index of the light-transmissive layer is n_{eff}, and a wavelength of the excitation light is λᵢ [nm], a relationship of the following expression (1) is satisfied.

20 [nm]<G·(n_{eff}/1.46)≦140 [nm]·(λᵢ/785 [nm]) (1)

### BACKGROUND

1. Technical Field

The present invention relates to an analysis apparatus and an electronic device.

2. Related Art

Recently, a demand for medical diagnosis, food inspection, or the like has increased greatly, and there has been a need to develop a compact and high-speed sensing technology. Various sensors commencing with an electrochemical method have been considered, and an interest with respect to a sensor using a surface plasmon resonance (SPR) has increased because integration is possible, the cost is reduced, and any measurement environment may be used. For example, a technology which detects a presence or absence of adsorption of a substance such as a presence or absence of adsorption of an antigen in an antigen-antibody reaction by using surface plasmon generated in a metallic thin film disposed on a total reflection prism surface has been known.

In addition, a method is also considered in which Raman scattering of a substance attached to a sensor portion is detected by using surface enhanced Raman scattering (SERS), and the attached substance is determined. SERS is a phenomenon in which Raman scattering light is enhanced 10² to 10¹⁴ times in a surface of metal in a nanometer scale. When a target substance which is in a state of being adsorbed onto the surface is irradiated with excitation light such as laser, light (Raman scattering light) having a wavelength which is slightly shifted from a wavelength of the excitation light by vibration energy of the substance (molecules) is scattered. When the scattering light is subjected to spectroscopic processing, a spectrum (a fingerprint spectrum) inherent to a type of substance (molecular species) is obtained. By analyzing a position or a shape of the fingerprint spectrum, it is possible to determine the substance with extremely high sensitivity.

It is preferable that such a sensor has a great enhancement degree of light on the basis of surface plasmon excited by light irradiation.

For example, in JP-T-2007-538264, a mutual interaction between localized surface plasmon (LSP) and surface plasmon polariton (SPP) is disclosed, and some parameters of a gap type surface plasmon polariton (GSPP) model are disclosed.

The GSPP of JP-T-2007-538264 has a dimension in which a size of particles causing a plasmon resonance is 50 nm to 200 nm, a periodic interparticle interval is shorter than an excitation wavelength, and a thickness of a dielectric body separating a particle layer from a mirror layer is 2 nm to 40 nm, and is in a regular array of plasmon resonance particles which are densely filled by an interparticle interval obtained by adding 0 nm to 20 nm to a particle dimension.

However, in a sensor having a structure disclosed in JP-T-2007-538264, it is found that a peak of an electric field enhancement degree in wavelength dependent properties (an enhancement degree spectrum or a reflectance spectrum) is broad, but an enhancement degree which is totally low and insufficient is obtained. In addition, in the sensor disclosed in JP-T-2007-538264, when a dimension of a plurality of particles is uneven (when a variation occurs), a wavelength having a peak in the enhancement degree spectrum is greatly shifted.

### SUMMARY

An advantage of some aspects of the invention is to provide an analysis apparatus and an electronic device in which a high enhancement degree is obtained in an enhancement degree spectrum, and a target substance is able to be detected and analyzed with high sensitivity. Another advantage of some aspects of the invention is to provide an analysis apparatus and an electronic device in which the target substance is easily attached to a position having a high enhancement degree. Still another advantage of some aspects of the invention is to provide an analysis apparatus and an electronic device in which an allowable range of a variation in manufacturing is wide.

The invention can be implemented as the following aspects or application examples.

An aspect of the invention is directed to an analysis apparatus including an electric field enhancing element including a metallic layer, a light-transmissive layer which is disposed on the metallic layer and transmits excitation light, and a plurality of metallic particles which is disposed on the light-transmissive layer, and is arranged in a first direction and a second direction intersecting with the first direction; a light source irradiating the electric field enhancing element with at least one of linearly polarized light which is polarized in the first direction, linearly polarized light which is polarized in the second direction, and circularly polarized light as the excitation light; and a detector detecting light emitted from the electric field enhancing element, in which localized surface plasmon excited to the metallic particles and propagating surface plasmon excited to a surface boundary between the metallic layer and the light-transmissive layer are electromagnetically and mutually interacted, and when a thickness of the light-transmissive layer is G [nm], an effective reflective index of the light-transmissive layer is n_{eff}, and a wavelength of the excitation light is λᵢ [nm], a relationship of the following expression (1) is satisfied.

20 [nm]<G·(n_{eff}/1.46)≦140 [nm]·(λᵢ/785 [nm]) (1)

According to the analysis apparatus, an extremely high enhancement degree is obtained in an enhancement degree spectrum, and a target substance is able to be detected and analyzed with high sensitivity. In addition, a position in which a high enhancement degree of the analysis apparatus is obtained exists on at least an upper surface side of metallic particles, and thus the target substance is easily in contact with the position, and it is possible to detect and analyze the target substance with high sensitivity. Further, this analysis apparatus satisfies a relationship of 40 [nm]≦G·(n_{eff}/1.46), and thus it is possible to increase an allowable range of a variation in manufacturing.

Another aspect of the invention is directed to an analysis apparatus including an electric field enhancing element including a metallic layer, a light-transmissive layer which is disposed on the metallic layer and transmits excitation light, and a plurality of metallic particles which is disposed on the light-transmissive layer, and is arranged in a first direction and a second direction intersecting with the first direction; a light source irradiating the electric field enhancing element with at least one of linearly polarized light which is polarized in the first direction, linearly polarized light which is polarized in the second direction, and circularly polarized light as the excitation light; and a detector detecting light emitted from the electric field enhancing element, in which localized surface plasmon excited to the metallic particles and propagating surface plasmon excited to a surface boundary between the metallic layer and the light-transmissive layer are electromagnetically and mutually interacted, the light-transmissive layer is formed of a laminated body in which m layers are laminated, m is a natural number, the light-transmissive layer is formed by laminating a first light-transmissive layer, a second light-transmissive layer, . . . , a (m−1)-th light-transmissive layer, and a m-th light-transmissive layer in this order from the metallic particle side to the metallic layer side, and when a refractive index in the vicinity of the metallic particles is n₀, an angle between a normal direction of the metallic layer and an incident direction of the excitation light is θ₀, an angle between the normal direction of the metallic layer and an incident direction of refracting light of the excitation light in the m-th light-transmissive layer with respect to the metallic layer is θₘ, a refractive index of the m-th light-transmissive layer is nₘ, a thickness of the m-th light-transmissive layer is Gₘ [nm], and a wavelength of the excitation light is λᵢ [nm], relationships of the following expression (2) and expression (3) are satisfied.

$$\begin{matrix} {n}_{0} \cdot sin {θ}_{0} = {n}_{m} \cdot sin {θ}_{m} & ( 2 ) \\ 20 [ nm ] < {\sum }_{m = 1}^{m} \{ ( {G}_{m} \cdot cos {θ}_{m} ) \cdot ( {n}_{m} / 1.46 ) \} ≦ 140 [ nm ] \cdot {λ}_{i} / 785 [ nm ] & ( 3 )\end{matrix}$$

According to the analysis apparatus, an extremely high enhancement degree is obtained in an enhancement degree spectrum, and a target substance is able to be detected and analyzed with high sensitivity. In addition, a position in which a high enhancement degree of the analysis apparatus is obtained exists on at least an upper surface side of metallic particles, and thus the target substance is easily in contact with the position, and it is possible to detect and analyze the target substance with high sensitivity. Further, this analysis apparatus satisfies a relationship:

$$40 [ nm ] ≦ {\sum }_{m = 1}^{m} \{ ( {G}_{m} \cdot cos {θ}_{m} ) \cdot ( {n}_{m} / 1.46 ) \}$$

and thus it is possible to increase an allowable range of a variation in manufacturing.

In the analysis apparatus according to the aspect of the invention, a first pitch P1 at which the metallic particles are arranged in the first direction, and a second pitch P2 at which the metallic particles are arranged in the second direction may be identical to each other.

According to the analysis apparatus with this configuration, an extremely high enhancement degree is obtained in an enhancement degree spectrum, and a target substance is able to be detected and analyzed with high sensitivity.

Still another aspect of the invention is directed to an analysis apparatus including an electric field enhancing element including a metallic layer, a light-transmissive layer which is disposed on the metallic layer and transmits excitation light, and a plurality of metallic particles which is disposed on the light-transmissive layer, and is arranged in a first direction at a first pitch and arranged in a second direction intersecting with the first direction at a second pitch; a light source irradiating the electric field enhancing element with at least one of linearly polarized light which is polarized in the first direction, linearly polarized light which is polarized in the second direction, and circularly polarized light as the excitation light; and a detector detecting light emitted from the electric field enhancing element, in which arrangement of the metallic particles of the electric field enhancing element satisfies a relationship of the following expression (4):

P1<P2≦Q+P1 (4)

[in which P1 is the first pitch, P2 is the second pitch, and Q is a pitch of a diffraction grating satisfying the following expression (5) when an angular frequency of localized plasmon excited to a row of the metallic particles is ω, a dielectric constant of metal configuring the metallic layer is ∈ (ω), a dielectric constant in the vicinity of the metallic particles is ∈, a speed of light in vacuum is c, and an inclined angle from a thickness direction of the metallic layer which is an irradiation angle of the excitation light is θ:

(ω/c)·{∈·∈(ω)/(∈+∈(ω))}^{1/2}=∈^{1/2}·(ω/c)·sin θ+2aπ/Q(a=±1,±2, . . . ) (5)], and

when a thickness of the light-transmissive layer is G [nm], an effective reflective index of the light-transmissive layer is n_{eff}, and a wavelength of the excitation light is λᵢ [nm], a relationship of the following expression (1) is satisfied:

20 [nm]<G·(n_{eff}/1.46)≦140 [nm]·(λᵢ/785 [nm]) (1).

In the analysis apparatus according to the aspect of the invention, the first pitch P1 may satisfy a relationship of 60 [nm]≦P1≦1310 [nm].

In the analysis apparatus according to the aspect of the invention, the second pitch P2 may satisfy a relationship of 60 [nm]≦P2≦1310 [nm].

In the analysis apparatus according to the aspect of the invention, the light-transmissive layer may include a layer selected from silicon oxide, titanium oxide, aluminum oxide, silicon nitride, and tantalum oxide.

In the analysis apparatus according to the aspect of the invention, the metallic layer may include a layer formed of gold, silver, copper, platinum, or aluminum.

In the analysis apparatus according to the aspect of the invention, a ratio of intensity of localized surface plasmon excited to a corner portion of the metallic particles on a side away from the light-transmissive layer to intensity of localized surface plasmon excited to a corner portion of the metallic particles on a side close to the light-transmissive layer may be constant regardless of the thickness of the light-transmissive layer.

In this case, according to the analysis apparatus, even when the thickness of the light-transmissive layer varies, the ratio of the intensity of the localized surface plasmon excited to an upper surface side of the metallic particles to the intensity of the localized surface plasmon excited to a lower surface side of the metallic particles does not vary, and thus the analysis apparatus is more easily manufactured.

Yet another aspect of the invention is directed to an electronic device including the analysis apparatus described above; a calculation unit which calculates medical health information on the basis of detection information from the detector; a storage unit which stores the medical health information; and a display unit which displays the medical health information.

According to the electronic device, an enhancement degree is extremely high, and a target substance is able to be detected and analyzed with high sensitivity, and thus medical health information with high sensitivity and high accuracy is able to be provided.

### BRIEF DESCRIPTION OF THE DRAWINGS

The invention will be described with reference to the accompanying drawings, wherein like numbers reference like elements.

FIG. 1 is a perspective view schematically illustrating a main part of an electric field enhancing element according to an embodiment.

FIG. 2 is a schematic view of the main part of the electric field enhancing element according to the embodiment seen in a plan view.

FIG. 3 is a schematic view of a cross-sectional surface of the main part of the electric field enhancing element according to the embodiment.

FIG. 4 is a schematic view of the cross-sectional surface of the main part of the electric field enhancing element according to the embodiment.

FIG. 5 is a schematic view illustrating an example of a light path of excitation light.

FIG. 6 is a schematic view illustrating an example of the light path of the excitation light.

FIG. 7 is a dispersion relationship according to a refractive index in the vicinity of a metallic layer.

FIG. 8 is a wavelength characteristic of a dielectric constant of silver.

FIG. 9 is a diagram illustrating a dispersion relationship and an electromagnetic coupling between propagating surface plasmon of the metallic layer and localized surface plasmon of metallic particles.

FIG. 10 is a schematic view of an analysis apparatus according to the embodiment.

FIG. 11 is a schematic view of an electronic device according to the embodiment.

FIG. 12 is a schematic view of a model according to an experimental example.

FIG. 13 is an example of a reflectance spectrum (far-field properties).

FIG. 14 is a reflectance spectrum and SQRT of the model according to the experimental example.

FIG. 15A is the reflectance spectrum of the model according to the experimental example.

FIG. 15B is the reflectance spectrum of the model according to the experimental example.

FIG. 16 is a graph illustrating dependent properties of a wavelength having a peak in a reflectance spectrum and a minimum value of the peak in the reflectance spectrum in the model according to the experimental example with respect to a thickness G of a light-transmissive layer.

FIGS. 17A and 17B are graphs illustrating light-transmissive layer thickness dependent properties of SQRT and a top/bottom ratio of the model according to the experimental example.

FIG. 18 shows graphs illustrating the dependent properties of the wavelength having a peak in the reflectance spectrum and the minimum value of the peak in the reflectance spectrum in the model according to the experimental example with respect to the thickness G of the light-transmissive layer.

FIG. 19 shows graphs illustrating the light-transmissive layer thickness dependent properties of SQRT of the model according to the experimental example.

FIG. 20 shows graphs illustrating light-transmissive layer thickness dependent properties of a minimum wavelength having a peak in the reflectance spectrum of the model according to the experimental example.

FIG. 21 is the reflectance spectrum of the model according to the experimental example.

FIGS. 22A and 22B are graphs illustrating the light-transmissive layer thickness dependent properties of SQRT of the model according to the experimental example.

FIG. 23 shows graphs illustrating light-transmissive layer thickness dependent properties of a minimum wavelength having a peak in the reflectance spectrum and reflectance of the model according to the experimental example.

FIGS. 24A to 24C are maps illustrating E_{z} in XZ (X pitch/4, 0, 0) of the model according to the experimental example.

FIGS. 25A to 25D are graphs comparing light-transmissive layer thickness dependence properties of PSP, LSP, PSP*LSP (a product of PSP and LSP), and SQRT of the model according to the experimental example.

FIG. 26 is a schematic view illustrating a relationship between the arrangement of the metallic particles and LSP and PSP.

### DESCRIPTION OF EXEMPLARY EMBODIMENTS

Hereinafter, some embodiments of the invention will be described. The following embodiments describe an example of the invention. The invention is not limited to the following embodiments, and includes various modifications performed within a range not changing the gist of the invention. Furthermore, all of the following configurations are not an essential configuration of the invention.

### 1. ELECTRIC FIELD ENHANCING ELEMENT

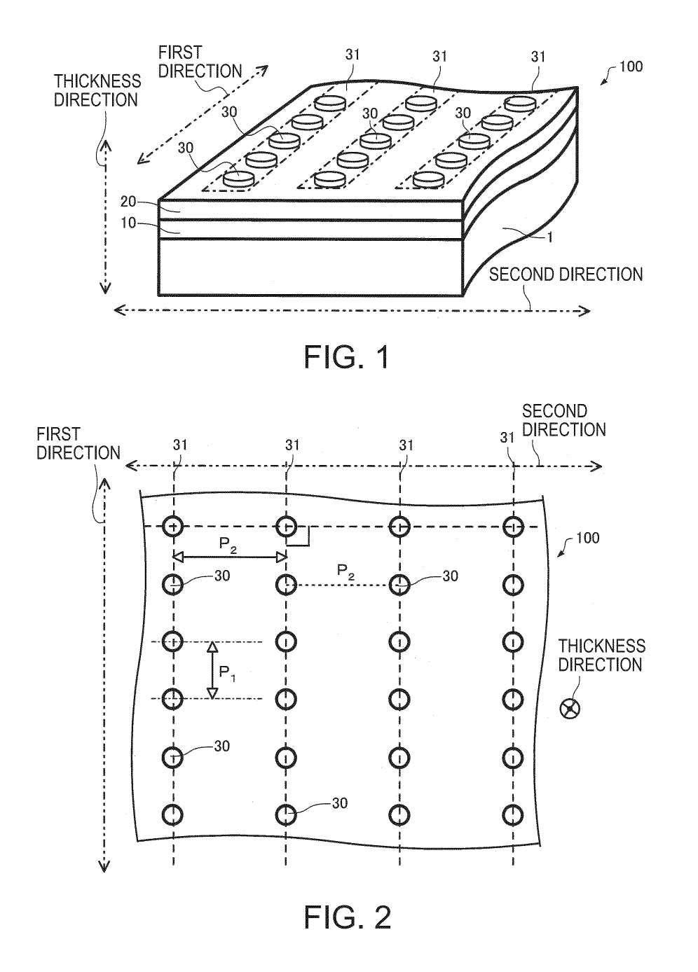

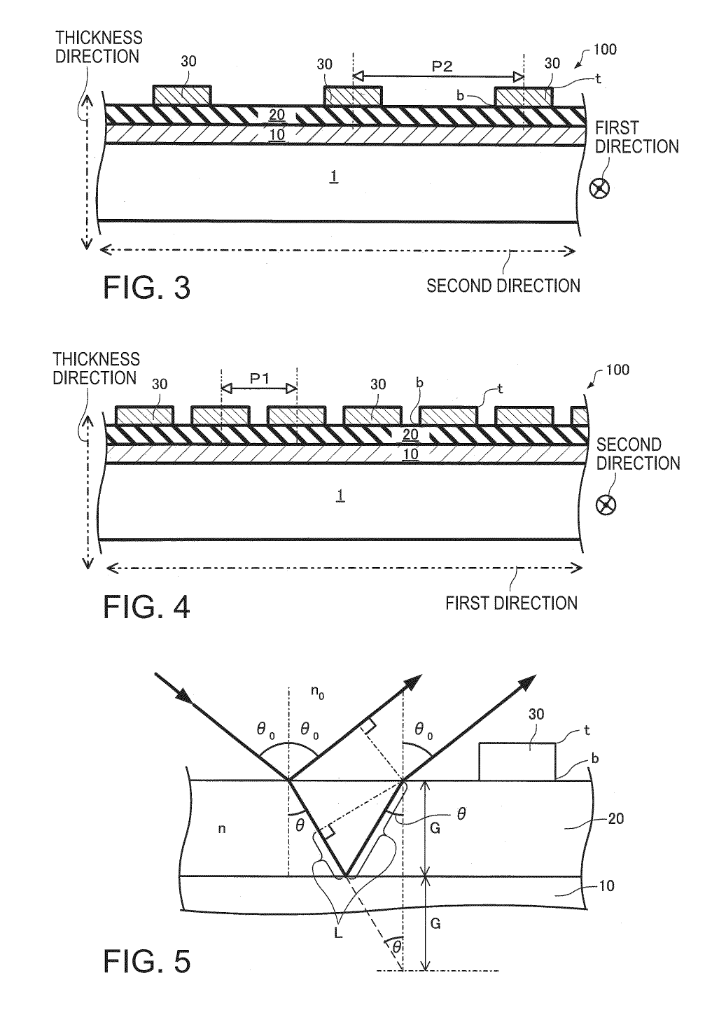

FIG. 1 is a perspective view of an electric field enhancing element 100 according to an example of an embodiment. FIG. 2 is a schematic view of the electric field enhancing element 100 according to an example of the embodiment seen in a plan view (seen from a thickness direction of a light-transmissive layer). FIG. 3 and FIG. 4 are schematic views of a cross-sectional surface of the electric field enhancing element 100 according to an example of the embodiment. The electric field enhancing element 100 of this embodiment includes a metallic layer 10, a light-transmissive layer 20, and metallic particles 30.

### 1.1. Metallic Layer

The metallic layer 10 is not particularly limited insofar as a surface of metal is provided, and for example, may be in the shape of a thick plate, a film, a layer, or a membrane. The metallic layer 10, for example, may be disposed on a substrate 1. In this case, the substrate 1 is not particularly limited, and as the substrate 1, a substrate which does not have an influence on propagating surface plasmon excited to the metallic layer 10 is preferable. As the substrate 1, for example, a glass substrate, a silicon substrate, a resin substrate, and the like are included. A shape of a surface of the substrate 1 on which the metallic layer 10 is disposed is not particularly limited. When a regular structure is formed on a surface of the metallic layer 10, the surface may correspond to the regular structure, and when the surface of the metallic layer 10 is a flat surface, the surface of the substrate 1 may be a flat surface. In examples of FIG. 1 to FIG. 4, the metallic layer 10 is disposed on the surface (a flat surface) of the substrate 1.

Here, an expression of the flat surface does not indicate a mathematically strict flat surface which is flat (smooth) without having a few concavities and convexities. For example, when there are concavities and convexities due to a constituent atom, concavities and convexities due to a secondary structure (crystal, grain aggregation, a grain boundary, and the like) of a constituent substance, or the like in the surface, the surface may not be strictly a flat surface from a microscopic viewpoint. However, even in this case, from a macroscopic viewpoint, the concavities and convexities are not remarkable, and are observed to the extent of not having difficulty in referring to the surface as a flat surface. Therefore, herein, insofar as a flat surface is able to be recognized from such a macroscopic viewpoint, a surface is referred to as a flat surface.

In addition, in this embodiment, a thickness direction of the metallic layer 10 is identical to a thickness direction of the light-transmissive layer 20 described later. Herein, when the thickness direction of the metallic layer 10 or the thickness direction of the light-transmissive layer 20 is described with respect to the metallic particles 30 described later, or the like, the thickness direction may be referred to as a thickness direction, a height direction, and the like. In addition, for example, when the metallic layer 10 is disposed on the surface of the substrate 1, a normal direction of the surface of the substrate 1 may be referred to as a thickness direction, a thickness direction or a height direction.

The metallic layer 10, for example, is able to be formed by a method such as vapor deposition, sputtering, casting, and machining. When the metallic layer 10 is disposed on the substrate 1, the metallic layer 10 may be disposed on the entire surface of the substrate 1, or may be disposed on a part of the surface of the substrate 1. A thickness of the metallic layer 10 is not particularly limited insofar as propagating surface plasmon is able to be excited to the surface of the metallic layer 10, or the vicinity of a surface boundary between the metallic layer 10 and the light-transmissive layer 20, and for example, is able to be greater than or equal to 10 nm and less than or equal to 1 mm, preferably greater than or equal to 20 nm and less than or equal to 100 μm, and more preferably greater than or equal to 30 nm and less than or equal to 1 μm.

The metallic layer 10 is formed of metal having an electric field applied by excitation light, and an electric field in which polarization induced by the electric field is vibrated in an antiphase, that is, metal capable of having a dielectric constant in which a real part of a dielectric function is a negative value (a negative dielectric constant), and a dielectric constant of an imaginary part is smaller than an absolute value of a dielectric constant of the real part when a specific electric field is applied. As an example of metal capable of having such a dielectric constant, gold, silver, aluminum, copper, platinum, an alloy thereof, and the like are able to be included. When light in a visible light region is used as the excitation light, it is preferable that the metallic layer 10 includes a layer formed of gold, silver, or copper among the metals. In addition, the surface of the metallic layer 10 (an end surface in the thickness direction) may not be a specific crystal plane. In addition, the metallic layer 10 may be formed of a plurality of metallic layers.

The metallic layer 10 has a function of generating the propagating surface plasmon in the electric field enhancing element 100 of this embodiment. Light is incident on the metallic layer 10 under a condition described later, and thus the propagating surface plasmon is generated in the vicinity of the surface of the metallic layer 10 (an end surface of the thickness direction). In addition, herein, quantum of vibration of an electric charge in the vicinity of the surface of the metallic layer 10 and vibration to which an electromagnetic wave is bonded is referred to as surface plasmon polariton (SPP). The propagating surface plasmon generated in the metallic layer 10 is able to mutually interact (hybrid) with localized surface plasmon generated in the metallic particles 30 described later in a constant condition. Further, the metallic layer 10 has a function of a mirror reflecting light (for example, refracting light of the excitation light) toward the light-transmissive layer 20 side.

### 1.2. Light-Transmissive Layer

The electric field enhancing element 100 of this embodiment includes the light-transmissive layer 20 for separating the metallic layer 10 from the metallic particles 30. In FIG. 1, FIG. 3, and FIG. 4, the light-transmissive layer 20 is illustrated. The light-transmissive layer 20 is able to be in the shape of a film, a layer, or a membrane. The light-transmissive layer 20 is disposed on the metallic layer 10. Accordingly, it is possible to separate the metallic layer from the metallic particles 30. In addition, the light-transmissive layer 20 is able to transmit the excitation light.

The light-transmissive layer 20, for example, is able to be formed by a method such as vapor deposition, sputtering, CVD, and various coatings. The light-transmissive layer 20 may be disposed on the entire surface of the metallic layer 10, or may be disposed on a part of the surface of the metallic layer 10.

The light-transmissive layer 20 may have a positive dielectric constant, and for example, is able to be formed of silicon oxide (SiOₓ, for example, SiO₂), aluminum oxide (AlₓO_{y}, for example, Al₂O₃), tantalum oxide (Ta₂O₅), silicon nitride (Si₃N₄), titanium oxide (TiOₓ, for example, TiO₂), high molecules such as a Polymethylmethacrylate (PMMA), Indium Tin Oxide (ITO), and the like. In addition, the light-transmissive layer 20 is able to be formed of a dielectric body. Further, the light-transmissive layer 20 may be configured of a plurality of layers having materials which are different from each other.

A thickness G of the light-transmissive layer 20 is set such that the propagating surface plasmon of the metallic layer 10 is able to mutually interact with the localized surface plasmon of the metallic particles 30. For example, the thickness G [nm] of the light-transmissive layer 20 is set as follows.

(i) When an effective refractive index of the light-transmissive layer 20 is n_{eff}, and a wavelength of the excitation light is λᵢ [nm], the thickness G [nm] of the light-transmissive layer 20 is set to satisfy a relationship of the following expression (1).

20 [nm]<G·(n_{eff}/1.46)≦140 [nm]˜(λᵢ/785 [nm]) (1)

Here, when the light-transmissive layer 20 is formed of a single layer, the effective refractive index n_{eff} of the light-transmissive layer 20 is identical to a value of a refractive index of a material configuring the single layer. In contrast, when the light-transmissive layer 20 is formed of a plurality of layers, the effective refractive index n_{eff} of the light-transmissive layer 20 is identical to a value obtained by dividing a product of a thickness of each layer configuring the light-transmissive layer 20 and a refractive index of each layer by the entire thickness G of the light-transmissive layer 20.

FIG. 5 is a diagram schematically illustrating a light path of the excitation light when the light-transmissive layer 20 is configured of a single layer having a refractive index n. With reference to FIG. 5, in a case where the light-transmissive layer 20 is configured of the single layer having a refractive index n, when the excitation light inclines at an inclined angle θ₀ with respect to a normal direction (the thickness direction) of the light-transmissive layer 20 from a phase having a refractive index of n₀, and is incident on the light-transmissive layer 20, the refracting light of the excitation light satisfying a relationship of n₀·sin θ₀=n·sin θ from Snell's law is generated in the light-transmissive layer 20 at the inclined angle θ with respect to the normal direction of the light-transmissive layer 20 (in the expression, “·” indicates a product).

Then, a light path difference between light reflected by an upper surface of the light-transmissive layer and light reflected by a lower surface of the light-transmissive layer 20 is 2·n·G·cos θ (refer to FIG. 5). In addition, a half-wavelength is shifted due to the reflection by the metallic layer 10, and thus when the wavelength of the excitation light is λᵢ, the light path difference is k·λᵢ (here, k is an integer). Accordingly, 2·n·G·cos θ=k·λᵢ is completed, and a relationship of sin θ=(n₀/n)·sin θ₀ and θ=sin⁻¹ {(n₀/n) sin θ₀} is completed.

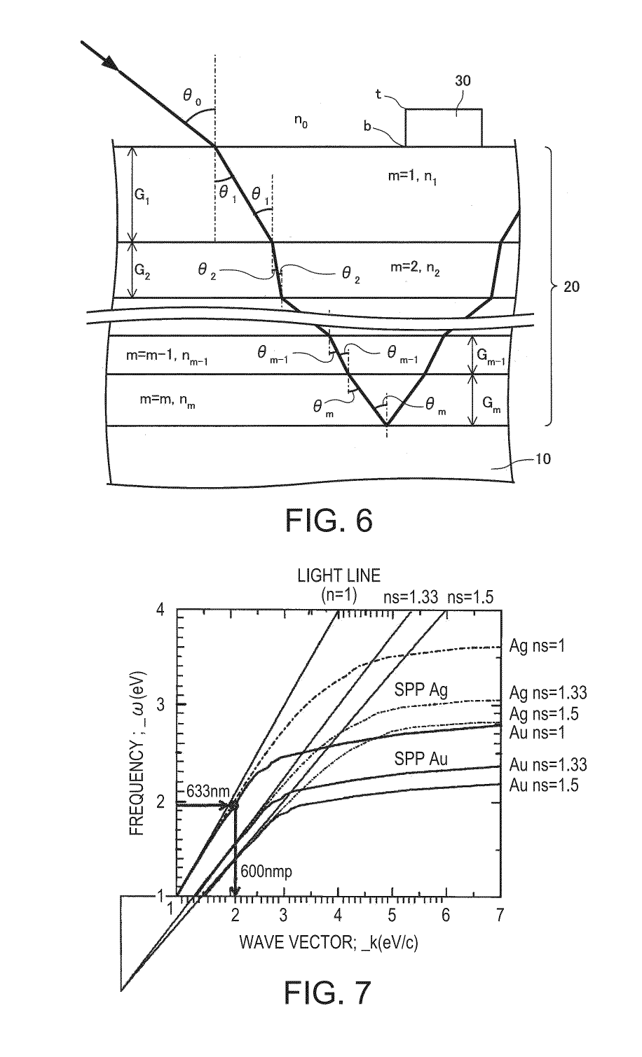

(ii) FIG. 6 is a diagram schematically illustrating the light path of the excitation light when the light-transmissive layer 20 is configured of a plurality of layers. With reference to FIG. 6, in a case where the light-transmissive layer 20 is configured of the plurality of layers, when the excitation light inclines at the inclined angle θ₀ with respect to the normal direction (the thickness direction) of the light-transmissive layer 20, and is incident on the light-transmissive layer 20, the light-transmissive layer 20 is considered as a light-transmissive layer in which a first light-transmissive layer, a second light-transmissive layer, a (m−1)-th light-transmissive layer, and a m-th light-transmissive layer are laminated in this order from a side away from the metallic layer 10 toward the metallic layer 10 (here, m is an integer greater than or equal to 2). Then, the excitation light inclines at the inclined angle θ₀ with respect to the normal direction (the thickness direction) of the light-transmissive layer 20 from the phase having a refractive index of n₀, and is incident on the light-transmissive layer 20. In this case, when an angle between the normal direction of the light-transmissive layer 20 and the refracting light of the excitation light in the m-th light-transmissive layer is θₘ, a refractive index of the m-th light-transmissive layer is nₘ, and a thickness of the m-th light-transmissive layer is Gₘ [nm], the refracting light of the excitation light satisfying a relationship of n₀·sin θ₀=nₘ·sin θₘ from Snell's law is generated in the m-th light-transmissive layer at the inclined angle θₘ with respect to the normal direction of the light-transmissive layer 20. Accordingly, when the thickness of the m-th light-transmissive layer is Gₘ, and the refractive index of the m-th light-transmissive layer is nₘ, a light path difference of 2·nₘ·Gₘ·cos θₘ is generated in each layer.

According to this, a total light path difference L is L=Σ(2·nₘ·Gₘ·cos θₘ). Then, when the light path difference L is an integer times (k·λᵢ) a wavelength of incident light, the light is intensified. In addition, it is understood that in a case of a vertical incidence (an incident direction of the excitation light is parallel with the thickness direction of the light-transmissive layer 20), θ₀ is 0, and a value of cos θₘ is 1, and in a case of an oblique incidence, a value of cos θₘ is smaller than 1, and thus a thickness Gₘ in which light is intensified is greater (thicker) in the oblique incidence than in the vertical incidence.

In addition, when the light-transmissive layer 20 is formed of a laminated body in which m layers are laminated (m is a natural number), the thickness G of the light-transmissive layer 20 is considered as the light-transmissive layer 20 in which the first light-transmissive layer, the second light-transmissive layer, the (m−1)-th light-transmissive layer, and the m-th light-transmissive layer are laminated from the side away from the metallic layer 10 toward the metallic layer 10. Then, the excitation light inclines at the inclined angle θ₀ with respect to the normal direction (the thickness direction) of the light-transmissive layer 20 from the phase having a refractive index of n₀, and is incident on the light-transmissive layer 20. In this case, the angle between the normal direction of the light-transmissive layer 20 and the refracting light of the excitation light in the m-th light-transmissive layer is θₘ, the refractive index of the m-th light-transmissive layer is nₘ, and the thickness of the m-th light-transmissive layer is Gₘ [nm], the refracting light of the excitation light satisfying a relationship of n₀·sin θ₀=nₘ·sin θₘ from Snell's law is generated in the m-th light-transmissive layer at the inclined angle θₘ with respect to the normal direction of the light-transmissive layer 20.

Then, when the wavelength of the excitation light is λᵢ [nm], relationships of the following expressions (2) and (3) are satisfied.

$$\begin{matrix} {n}_{0} \cdot sin {θ}_{0} = {n}_{m} \cdot sin {θ}_{m} & ( 2 ) \\ 20 [ nm ] < {\sum }_{m = 1}^{m} \{ ( {G}_{m} \cdot cos {θ}_{m} ) \cdot ( {n}_{m} / 1.46 ) \} ≦ 140 [ nm ] \cdot {λ}_{i } / 785 [ nm ] & ( 3 )\end{matrix}$$

In the expressions (i) and (ii) described above, all of “20 [nm]”, “140 [nm]”, “785 [nm]”, and “1.46 [−] (a dimensionless number)” are empirical values which are experimentally obtained by consideration of the inventors, and one of important parameters of the invention. The thickness G of the light-transmissive layer 20 is set by any one method of (i) and (ii) described above, and thus an electric field enhancement degree of the electric field enhancing element 100 of this embodiment extremely increases.

A lower limit value of the expressions (1) and (3) described above is 20 nm because it is a value empirically obtained to be verified by an experimental example described later. In addition, (λᵢ/785 [nm]) multiplied by an upper limit value of the expressions (1) and (3) is a correction term for expressing that even when the wavelength of the excitation light is changed, each expression is completed. Further, (n/1.46) multiplied by G of the expressions (1) and (3) is a correction term for expressing that even when the refractive index of the light-transmissive layer is changed, each expression is completed. These correction terms are established by experimental examples described later.

Further, it is considered that a lower limit value in the expression (1) and (3) described above is 30 nm, 40 nm, and the like due to the following reasons. According to the structure of the electric field enhancing element 100 of this embodiment, a plurality of metallic particles 30 is disposed on the light-transmissive layer 20. When the thickness G of the light-transmissive layer 20 is below approximately 20 nm, a variation amount in a position of an enhancement degree peak in an electric field enhancing spectrum of the electric field enhancing element 100 extremely increases due to a variation in a size of the metallic particles 30. For example, as described in the following experimental examples, when the thickness G of the light-transmissive layer 20 is approximately 20 nm, a strong enhancement degree is obtained, but a peak position of an enhancement degree is sensitive to a change in a diameter of the metallic particles 30, and thus a design of an electric field enhancement degree profile of the electric field enhancing element 100 is slightly cumbersome. For this reason, on the contrary, the thickness G of the light-transmissive layer 20 may exceed 20 nm (20 nm<G), and more preferably, the thickness G of the light-transmissive layer 20 is greater than or equal to approximately 30 nm, and thus the electric field enhancing element 100 is easily designed, and it is possible to increase an allowable range of a variation in manufacturing.

Further, when the thickness G of the light-transmissive layer 20 is below approximately 40 nm, a mutual interaction between the localized surface plasmon in the vicinity of the metallic particles 30 and the propagating surface plasmon in the vicinity of the surface of the metallic layer 10 increases. As described in the following experimental examples, when the thickness G of the light-transmissive layer 20 is below approximately 40 nm, a ratio of an enhancement degree of a top of the metallic particles 30 to an enhancement degree in a bottom of the metallic particles 30 decreases. Thus, a distribution of energy for enhancing an electric field is biased to the bottom of the metallic particles 30, and thus usage efficiency of the energy of the excitation light for forming an enhanced electric field for detecting a trace substance decreases. Therefore, the thickness G of the light-transmissive layer 20 is greater than or equal to approximately 40 nm, and thus it is possible to more effectively use the energy of the excitation light for forming the enhanced electric field for detecting the trace substance. Furthermore, this will be described in “1.5. Position of Hot Spot” and the like.

### 1.3. Metallic Particles

The metallic particles 30 are disposed to be separated from the metallic layer 10 in the thickness direction. That is, the metallic particles 30 are disposed on the light-transmissive layer 20, and are arranged to be spatially separated from the metallic layer 10. The light-transmissive layer 20 is disposed between the metallic particles 30 and the metallic layer 10. In an example of the electric field enhancing element 100 in FIG. 1 to FIG. 4 of this embodiment, the light-transmissive layer 20 is disposed on the metallic layer 10, and the metallic particles 30 are formed thereon, and thus the metallic layer 10 and the metallic particles 30 are arranged to be separated from the light-transmissive layer in the thickness direction.

A shape of the metallic particles 30 is not particularly limited. For example, the shape of the metallic particles 30 is able to be in the shape of a circle, an ellipse, a polygon, an infinite form, or a combination thereof when projecting in the thickness direction of the metallic layer 10 and the light-transmissive layer 20 (in a plan view seen from the thickness direction), and is able to be in the shape of a circle, an ellipse, a polygon, an infinite form, or a combination thereof when projecting in a direction perpendicular to the thickness direction. In all examples of FIG. 1 to FIG. 4, the metallic particles 30 are illustrated as a cylinder having a center axis in the thickness direction of the light-transmissive layer 20, but the shape of the metallic particles 30 is not limited thereto.

A size T of the metallic particles 30 in the height direction indicates a length of a section in which the metallic particles 30 are able to be cut by a flat surface vertical to the height direction, and is greater than or equal to 1 nm and less than or equal to 100 nm. In addition, a size of the metallic particles 30 in the first direction perpendicular to the height direction indicates a length of a section in which the metallic particles 30 are able to be cut by a flat surface vertical to the first direction, and is greater than or equal to 5 nm and less than or equal to 200 nm. For example, when the shape of the metallic particles 30 is a cylinder having a center axis in the height direction, a size of the metallic particles 30 in the height direction (a height of the cylinder) is greater than or equal to 1 nm and less than or equal to 100 nm, preferably greater than or equal to 2 nm and less than or equal to 50 nm, more preferably greater than or equal to 3 nm and less than or equal to 30 nm, and further preferably greater than or equal to 4 nm and less than or equal to 20 nm. In addition, when the shape of the metallic particles 30 is a cylinder having a center axis in the height direction, a size of the metallic particles 30 in the first direction (a diameter of a bottom surface of the cylinder) is greater than or equal to 10 nm and less than or equal to 200 nm, preferably greater than or equal to 20 nm and less than or equal to 150 nm, more preferably greater than or equal to 25 nm and less than or equal to 100 nm, and further preferably greater than or equal to 30 nm and less than or equal to 72 nm.

The shape or a material of the metallic particles 30 is arbitrary insofar as the localized surface plasmon is generated due to the irradiation of the excitation light, and as the material capable of generating the localized surface plasmon due to light in the vicinity of visible light, gold, silver, aluminum, copper, platinum, an alloy thereof, and the like are able to be included.

The metallic particles 30, for example, are able to be formed by a method in which a thin film is formed by sputtering, vapor deposition, and the like, and then is patterned, a micro-contact printing method, a nanoimprint method, and the like. In addition, the metallic particles 30 are able to be formed by a colloid chemical method, and may be arranged in a position separated from the metallic layer 10 by a suitable method.

The metallic particles 30 have a function of generating the localized surface plasmon (LSP) in the electric field enhancing element 100 of this embodiment. The metallic particles 30 are irradiated with the excitation light, and thus the localized surface plasmon (LSP) is able to be generated in the vicinity of the metallic particles 30. The localized surface plasmon generated in the metallic particles 30 is able to be mutually interacted (hybrid) with the propagating surface plasmon (PSP) generated in the metallic layer 10 described above under a constant condition.

### 1.3.1. Arrangement of Metallic Particles

As illustrated in FIG. 1 to FIG. 4, the metallic particles 30 are configured of a plurality of parallel metallic particle rows 31. The metallic particles 30 are arranged in parallel with the first direction perpendicular to the thickness direction of the metallic layer 10 in the metallic particle row 31. In other words, the metallic particle row 31 has a structure in which a plurality of metallic particles 30 is arranged in the first direction perpendicular to the height direction. When metallic particles 30 have a longitudinal shape (an anisotropic shape), the first direction in which the metallic particles 30 are arranged may not be coincident with a longitudinal direction thereof. A plurality of metallic particles 30 may be arranged in one metallic particle row 31, and the number of arranged metallic particles 30 is preferably greater than or equal to 10.

Here, a pitch of the metallic particles 30 in the first direction inside the metallic particle row 31 is defined as a first pitch P1 (refer to FIG. 2 to FIG. 4). The first pitch P1 indicates a distance between gravity centers of two metallic particles 30 in the first direction. Furthermore, when the metallic particles 30 are in the shape of a cylinder having a center axis in the thickness direction of the metallic layer 10, an interparticle distance between two metallic particles 30 inside the metallic particle row 31 is identical to a length obtained by subtracting a diameter of the cylinder from the first pitch P1.

The first pitch P1 of the metallic particles 30 in the first direction inside the metallic particle row 31 is able to be greater than or equal to 10 nm and less than or equal to 2 μm, preferably greater than or equal to 20 nm and less than or equal to 1500 nm, more preferably greater than or equal to 30 nm and less than 1000 nm, and further preferably greater than or equal to 50 nm and less than 800 nm.

The metallic particle row 31 is configured of a plurality of metallic particles 30 arranged in the first direction at the first pitch P1, and a distribution, intensity, or the like of the localized surface plasmon generated in the metallic particles 30 also depends on the arrangement of the metallic particles 30. Therefore, the localized surface plasmon mutually interacted with the propagating surface plasmon generated in the metallic layer 10 may include not only localized surface plasmon generated in single metallic particle 30, but also localized surface plasmon considering the arrangement of the metallic particles 30 in the metallic particle row 31.

As illustrated in FIG. 1 to FIG. 4, the metallic particle row 31 is arranged in parallel with the second direction intersecting with the thickness direction of the metallic layer 10 and the first direction at a second pitch P2. A plurality of metallic particle rows 31 may be arranged, and the number of arranged metallic particle rows 31 is preferably greater than or equal to 10 rows.

Here, an interval between adjacent metallic particle rows 31 in the second direction is defined as the second pitch P2. The second pitch P2 indicates a distance between gravity centers of two metallic particle rows 31 in the second direction. In addition, when the metallic particle row 31 is configured of a plurality of rows 22, the second pitch P2 indicates a distance between a position of a gravity center of a plurality of rows 22 in the second direction and a position of a gravity center of a plurality of rows 22 of the adjacent metallic particle rows 31 in the second direction.

Similar to the first pitch P1, the second pitch P2 between the metallic particle rows 31 is able to be greater than or equal to 10 nm and less than or equal to 2 μm, preferably greater than or equal to 20 nm and less than or equal to 1500 nm, more preferably greater than or equal to 30 nm and less than 1000 nm, and further preferably greater than or equal to 50 nm and less than 800 nm.

In addition, the first pitch P1 and the second pitch P2 may be identical (similar) to each other, or may be different from each other. Here, “identical” and “similar”, for example, indicates “identical” and “similar” in a range allowing for a difference resulted from an accumulation of errors in manufacturing, or errors of measurement. In addition, as one of the aspects in which the first pitch P1 and the second pitch P2 are identical to each other, an aspect in which the metallic particles 30 are arranged in the shape of a two-dimensional square grating (a unit grating is a square) such that the metallic particles 30 are arranged in the first direction at the first pitch P1, and are arranged in the second direction perpendicular to the first direction at the second pitch P2 identical to the first pitch P1 is included. In addition, as one of the aspects in which the first pitch P1 and the second pitch P2 are identical to each other, an aspect in which the metallic particles 30 are arranged in the shape of a two-dimensional grating (a unit grating is a rhombus) such that the metallic particles 30 are arranged in the first direction at the first pitch P1, and are arranged in the second direction which is not perpendicular to the first direction but intersects with the first direction at the second pitch P2 identical to the first pitch P1 is included.

Furthermore, an angle between a line of the metallic particle row 31 extending in the first direction and a line connecting two metallic particles 30 which are closest to each other in two metallic particles 30 each belonging to the adjacent metallic particle rows 31 is not particularly limited, and may be a right angle. For example, the angle between two lines may be a right angle, or may not be a right angle. That is, when the arrangement of the metallic particles 30 seen from the thickness direction is in the shape of a two-dimensional grating having a position of the metallic particles 30 as a grating point, an irreducible basic unit grating may be in the shape of a rectangle, or may be in the shape of a parallelogram. In addition, when the angle between the line of the metallic particle row 31 extending in the first direction and the line connecting the two metallic particles 30 which are closest to each other in the two metallic particles 30 each belonging to the adjacent metallic particle rows 31 is not a right angle, a pitch between the two metallic particles 30 which are closest to each other in the two metallic particles 30 each belonging to the adjacent metallic particle rows 31 may be the second pitch P2.

### 1.3.2. Propagating Surface Plasmon and Localized Surface Plasmon

First, the propagating surface plasmon will be described. FIG. 7 is a graph of a dispersion relationship illustrating a dispersion curve of the excitation light, gold (a solid line), and silver (a broken line). In general, even when light is incident on a surface of metal at an incident angle θ (an irradiation angle θ) of 0 to 90 degrees, the propagating surface plasmon is not generated. For example, this is because when the metal is formed of Au, as illustrated in FIG. 7, a light line and a dispersion curve of SPP of Au do not have an intersecting point. In addition, even when a refractive index of a medium through which light passes is changed, SPP of Au is also changed according to a peripheral refractive index, and thus the light line and the dispersion curve do not have the intersecting point. In order to cause the propagating surface plasmon to have the intersecting point, a method in which a metallic layer is disposed on a prism as Kretschmann arrangement, and a wavenumber of the excitation light is increased by a refractive index of the prism, or a method in which a wavenumber of the light line is increased by a diffraction grating is used. Furthermore, FIG. 7 is a graph illustrating a so-called dispersion relationship (a vertical axis is an angular frequency [ω (eV)], and a horizontal axis is a wave vector [k (eV/c)]).

In addition, the angular frequency ω (eV) of the vertical axis in the graph of FIG. 7 has a relationship of λ [nm]=1240/ω (eV), and is able to be converted to a wavelength. In addition, the wave vector k (eV/c) of the horizontal axis in the graph of FIG. 7 has a relationship of k (eV/c)=2π·2/[λ [nm]/100]. Therefore, for example, when a diffraction grating interval is Q, and Q is 600 nm, k is 2.09 (eV/c). In addition, the irradiation angle θ is an inclined angle from the thickness direction of the metallic layer 10 or the light-transmissive layer 20, or the height direction of the metallic particles 30 in the irradiation angle θ of the excitation light.

FIG. 7 illustrates the dispersion curve of SPP of gold (Au) and silver (Ag), and in general, when an angular frequency of the excitation light incident on the surface of the metal is ω, a speed of light in vacuum is c, a dielectric constant of the metal configuring the metallic layer 10 is ∈ (ω), and a peripheral dielectric constant is ∈, the dispersion curve of SPP of the metal is given as an expression (A):

K_{SPP}ω/c[∈·∈(ω)/(∈+∈(ω))]^{1/2} (A).

On the other hand, the inclined angle from the thickness direction of the metallic layer 10 or the light-transmissive layer 20, or the height direction of the metallic particles 30 in the irradiation angle of the excitation light is θ, a wavenumber K of the excitation light passing through a virtual diffraction grating having an interval Q is expressed by an expression (B):

K=n·(ω/c)·sin θ+a·2π/Q(a=±1,±2, . . . ) (B),

and this relationship is illustrated as a straight line but not a curve on the graph of the dispersion relationship.

Furthermore, in the expression (B), n is a peripheral refractive index, and an extinction coefficient is κ, a real part ∈′ and an imaginary part ∈″ of a specific dielectric constant ∈ in a frequency of light are given as ∈′=n²·κ², and ∈″=2nκ, and when a peripheral substance is transparent, ∈ is a real number of κ to 0, and thus ∈ is n², and n is ∈^{1/2}

In the graph of the dispersion relationship, when the dispersion curve of SPP of the metal (the expression (A) described above) and the straight line of the diffracted light (the expression (B) described above) have the intersecting point, the propagating surface plasmon is excited. That is, when a relationship of K_{SPP}=K is completed, the propagating surface plasmon is excited to the metallic layer 10.

Therefore, the following expression (C) is obtained from the expressions (A) and (B) described above, and it is understood that when a relationship of the expression (C) is satisfied:

(ω/c)·{∈·∈(ω)/(∈+∈(ω))}^{1/2}=∈^{1/2}·(ω/c)·sin θ+2aπ/Q(a=±1,±2, . . . ) (C)

the propagating surface plasmon is excited to the metallic layer 10. In this cos θ, according to an example of SPP in FIG. 7, θ and m are changed, and thus a slope and/or a segment of the light line are able to be changed, and the straight line of the light line is able to intersect with the dispersion curve of SPP of Au.

Next, the localized surface plasmon will be described.

A condition in which the localized surface plasmon is generated in the metallic particles 30 by the real part of the dielectric constant is given as:

Real [∈(ω)]=−2∈ (D).

When the peripheral refractive index n is 1, ∈=n²−κ²=1, and thus Real [∈(ω)]=−2.

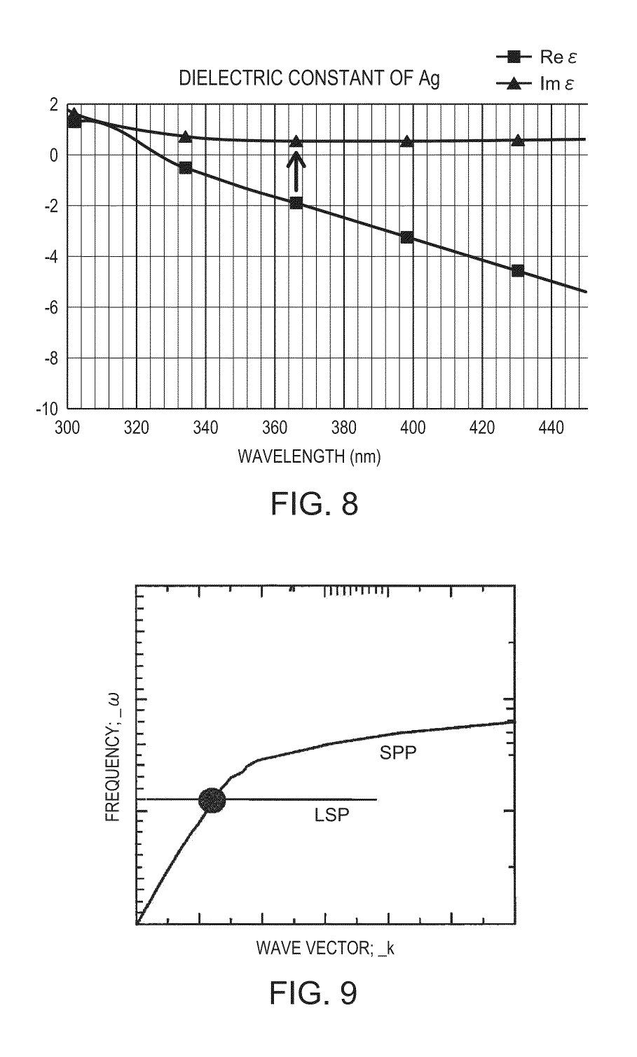

FIG. 8 is a graph illustrating a relationship between a dielectric constant of Ag and a wavelength. For example, the dielectric constant of Ag is as illustrated in FIG. 8, and the localized surface plasmon is excited at a wavelength of approximately 366 nm, but when a plurality of silver particles is close to a nano-order, or when silver particles and the metallic layer 10 (an Au film or the like) are arranged to be separated by the light-transmissive layer 20 (for example, SiO₂ or the like), an excitation peak wavelength of the localized surface plasmon is red-shifted (shifted to a long wavelength side) due to an influence of a gap thereof (the thickness G of the light-transmissive layer 20). A shift amount thereof depends on a dimension such as a diameter D of the silver particles, a thickness T of the silver particles, a particle interval between the silver particles, and the thickness G of the light-transmissive layer 20, and for example, exhibits a wavelength characteristic having a peak of the localized surface plasmon of 500 nm to 900 nm.

In addition, the localized surface plasmon is different from the propagating surface plasmon, and is plasmon which is not moved with a speed, and when plotting in the graph of the dispersion relationship, a slope is zero, that is, ω/k=0.

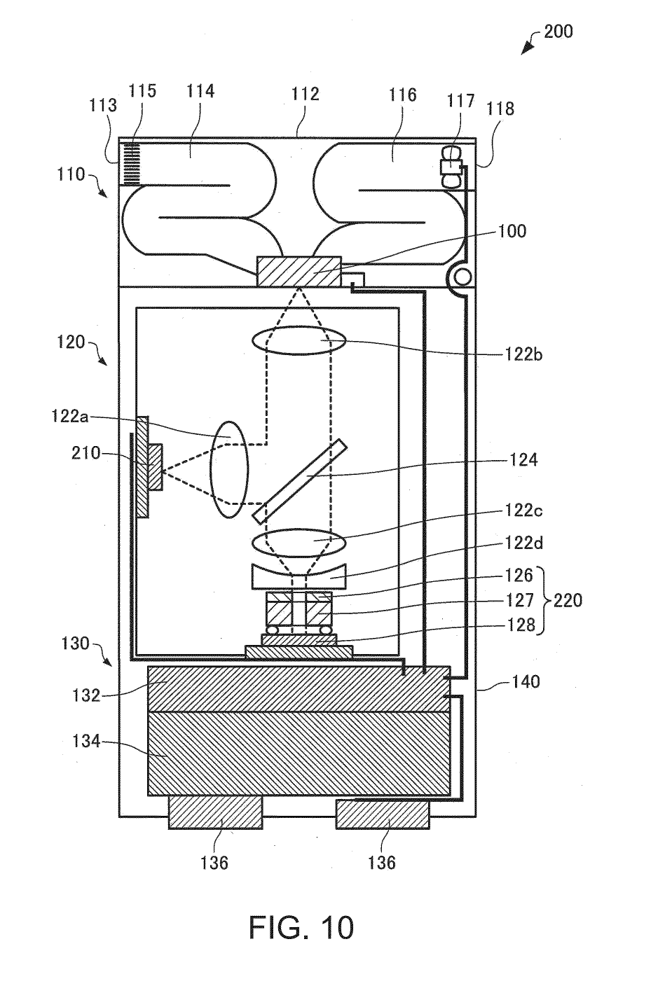

FIG. 9 is a diagram illustrating a dispersion relationship and an electromagnetic coupling between the surface plasmon polariton (SPP) of the metallic layer 10 and the localized surface plasmon (LSP) generated in the metallic particles 30. The electric field enhancing element 100 of this embodiment electromagnetically bonds (Electromagnetic Coupling) the propagating surface plasmon and the localized surface plasmon, and thus an enhancement degree having an extremely great electric field is obtained. That is, in the electric field enhancing element 100 of this embodiment, in the graph of the dispersion relationship, the intersecting point between the straight line of the diffracted light and the dispersion curve of SPP of the metal is not set as an arbitrary point, but the metallic particles 30 which are a diffraction grating are arranged such that the straight line of the diffracted light and the dispersion curve intersect with each other in the vicinity of a point in which the greatest or a maximum enhancement degree is obtained in the localized surface plasmon generated in the metallic particles 30 (the metallic particle row 31) (refer to FIG. 7 and FIG. 9). Therefore, in the electric field enhancing element 100 of this embodiment, the localized surface plasmon (LSP) excited to the metallic particles 30, and the propagating surface plasmon (PSP) excited to a surface boundary between the metallic layer 10 and the light-transmissive layer 20 are electromagnetically and mutually interacted. Furthermore, when the propagating surface plasmon and the localized surface plasmon are electromagnetically bonded (Electromagnetic Coupling), for example, anti-crossing behavior as described in OPTICS LETTERS/Vol. 34, No. 3/Feb. 1, 2009 or the like occurs.

In other words, in the electric field enhancing element 100 of this embodiment, it is designed such that the straight line of the diffracted light passes through the vicinity of an intersecting point between the dispersion curve of SPP of the metal and the angular frequency of the excitation light (a line in parallel with the horizontal axis of LSP in the graph of the dispersion relationship in FIG. 9) in which the greatest or the maximum enhancement degree is obtained in the localized surface plasmon generated in the metallic particles 30 (the metallic particle row 31) in the graph of the dispersion relationship.

### 1.3.2. Second Pitch P2

As described above, the second pitch P2 between the metallic particle rows 31 may be identical to the first pitch P1, or may be different from the first pitch P1, and for example, when the excitation light is in a vertical incidence (the incident angle θ=0), primary diffracted light (a=0) is used, and the interval Q of the diffraction grating described above is adopted as the second pitch P2, an expression (C) is able to be satisfied. However, the interval Q capable of satisfying the expression (C) has a width according to an incident angle and an order m of diffracted light to be selected. Furthermore, in this case, it is preferable that the incident angle θ is an inclined angle from the thickness direction to the second direction, and may be an inclined angle toward a direction including a component of the first direction.

Therefore, a range of the second pitch P2 in which a hybrid between the localized surface plasmon and the propagating surface plasmon is able to occur may satisfy a relationship of an expression (E) considering that the range is in the vicinity of the intersecting point described above (a width of ±P1).

Q−P1≦P2≦Q+P1 (E)

Furthermore, the second pitch P2 may satisfy a relationship of P1≦P2, and may satisfy a relationship of the following expression (F).

P1≦P2≦Q+P1 (F)

Furthermore, in general, in a case of a vertical incidence (in a case of an oblique incidence, a diffraction grating pitch passing through the intersecting point between LSP and SPP varies according to an incident angle, and thus the description thereof is inaccurate, and the vertical incidence will be described), when a value of the first pitch P1 and the second pitch P2 is smaller than the wavelength of the excitation light, intensity of the localized surface plasmon which is moved between the metallic particles 30 tends to increase, and on the contrary, when the value of the first pitch P1 and the second pitch P2 is close to the wavelength of the excitation light, intensity of the propagating surface plasmon generated in the metallic layer 10 tends to increase. Further, an electric field enhancement degree of the entire electric field enhancing element 100 depends on hot spot density (a rate of a region having a high electric field enhancement degree per unit area) (HSD), and thus HSD decreases as the value of the first pitch P1 and the second pitch P2 becomes greater. For this reason, the value of the first pitch P1 and the second pitch P2 is in a preferred range, and for example, it is preferable that the range is 60 nm P1≦1310 nm, and 60 nm P2≦1310 nm.

In addition, when P1=P2, it is preferable that both of P1 and P2 are approximately ±40% of the wavelength of the excitation light. Specifically, when the wavelength of the excitation light is 633 nm, and both of P1 and P2 are approximately 600 nm, an electric field enhancement degree increases. When the wavelength of the excitation light is 785 nm, and both of P1 and P2 are approximately 780 nm, the electric field enhancement degree increases.

### 1.4. Surface Enhanced Raman Scattering

The electric field enhancing element 100 of this embodiment indicates a high electric field enhancement degree. Therefore, the electric field enhancing element 100 is able to be preferably used for surface enhanced Raman scattering (SERS) measurement.

In Raman scattering, when a wavelength of excitation light is λᵢ, and a wavelength of scattering light is λₛ, a shift amount (cm⁻¹) due to the Raman scattering is given as the following expression (a).

Amount of Raman Scattering=(1/λᵢ)−(1/λₛ) (a)

Hereinafter, acetone will be described as an example of a target substance exhibiting a Raman scattering effect.

It is found that the acetone causes the Raman scattering in 787 cm⁻¹, 1708 cm⁻¹, and 2921 cm⁻¹.

According to the expression (a) described above, when the wavelength of excitation light λᵢ is 633 nm, the wavelength of stokes Raman scattering light λₛ due to acetone is 666 nm, 709 nm, and 777 nm each corresponding to the shift amount described above. In addition, when the wavelength of excitation light λᵢ is 785 nm, each wavelength λₛ is 837 nm, 907 nm, and 1019 nm corresponding to the shift amount described above.

In addition, there is also anti-strokes scattering, but in principle, an occurrence probability of the strokes scattering increases, and in the SERS measurement, strokes scattering in which a scattering wavelength is longer than an excitation wavelength is generally used.

On the other hand, in the SERS measurement, a phenomenon in which extremely low intensity of Raman scattering light is able to be dramatically increased by using an electric field enhancing effect due to surface plasmon is used. That is, an electric field enhancement degree Eᵢ of the wavelength of excitation light λᵢ and an electric field enhancement degree Eₛ of the wavelength of Raman scattering light λₛ are strong, HSD increases, and SERS intensity is proportionate to the following expression (b).

Eᵢ²·Eₛ²·HSD (b)

Here, Eᵢ represents the electric field enhancement degree of the wavelength of excitation light λᵢ, Eₛ represents the electric field enhancement degree of the wavelength of Raman scattering light λₛ, and HSD represents Hot Spot Density which is the number of hot spots per certain unit area.

That is, in the SERS measurement, it is preferable that a wavelength of excitation light to be used and a wavelength characteristic of Raman scattering light of a target substance to be detected are ascertained, and a wavelength of the excitation light, a wavelength of scattering light and a wavelength at a peak in an electric field enhancement degree (Reflectance) spectrum of surface plasmon are designed to be substantially coincident with one another in order that an SERS enhancement degree in proportion to the expression (b) described above is large. In addition, it is preferable that an SERS sensor has a broad peak in the electric field enhancement degree (reflectance) spectrum, and a value of a high enhancement degree.

In addition, when a surface plasmon resonance (SPR) is generated by the irradiation of the excitation light, absorption occurs due to the resonance, and the reflectance decreases. For this reason, intensity of an SPR enhanced electric field is able to be expressed by (1−r) using reflectance r. According to a relationship in which intensity of an enhanced electric field is strong as a value of the reflectance R becomes closer to zero, the reflectance is able to be used as an index of the intensity of the SPR enhanced electric field. For this reason, herein, it is considered that an enhancement degree profile (an enhancement degree spectrum) and a reflectance profile (a reflectance spectrum) are correlated with each other, the enhancement degree profile and the reflectance profile are regarded as identical to each other on the basis of the relationship described above.

### 1.5. Position of Hot Spot

When the electric field enhancing element 100 of this embodiment is irradiated with the excitation light, a region having a great enhanced electric field is generated at least in an end of the metallic particles 30 on an upper surface side, that is, a corner portion of the metallic particles 30 in a side away from the light-transmissive layer 20 (hereinafter, this position is referred to as a “top”, and is indicated by “t” in the drawings), and an end of the metallic particles on a lower surface side, that is, a corner portion of the metallic particles 30 on a side close to the light-transmissive layer 20 (hereinafter, this position is referred to as a “bottom”, and is indicated by “b” in the drawings). Furthermore, the corner portion of the metallic particles 30 on the side away from the light-transmissive layer 20 corresponds to a head portion of the metallic particles 30, and for example, indicates a peripheral portion of a surface (a circular surface) on the side away from the light-transmissive layer 20 when the metallic particles 30 are in the shape of a cylinder having a center axis in the normal direction of the light-transmissive layer 20. In addition, the corner portion of the metallic particles 30 on the side close to the light-transmissive layer 20 corresponds to a bottom portion of the metallic particles 30, and for example, indicates a peripheral portion of a surface (a circular surface) on the side close to the light-transmissive layer 20 when the metallic particles 30 are in the shape of a cylinder having a center axis in the normal direction of the light-transmissive layer 20.

It is considered that the metallic particles 30 are arranged on the light-transmissive layer 20 into a convex shape, and thus when a target substance is close to the electric field enhancing element 100, a probability of being in contact with the top of the metallic particles 30 is greater than a probability of being in contact with the bottom of the metallic particles 30.

In such a consideration, when focusing on a condition in which an electric field enhancement degree increases in the top of the metallic particles 30, it is possible to determine a range of the thickness G of the light-transmissive layer 20 described above. That is, as described above, the electric field enhancing element 100 of this embodiment includes the metallic layer 10, the light-transmissive layer 20 which is disposed on the metallic layer 10 and transmits the excitation light, and a plurality of metallic particles 30 which is disposed on the light-transmissive layer 20, and is arranged in the second direction intersecting with the first direction and the first direction, and at the time of the irradiation of the excitation light, the localized surface plasmon excited to the metallic particles 30 (neighborhood) and the propagating surface plasmon excited to the surface boundary (neighborhood) between the metallic layer 10 and the light-transmissive layer 20 are electromagnetically and mutually interacted. Then, by selecting the thickness G of the light-transmissive layer 20 according to at least one of the conditions (i) and (ii) described in “1.2. Light-Transmissive Layer”, it is possible to extremely increase an electric field enhancement degree in the top of the metallic particles 30.

In addition, according to the structure of the electric field enhancing element 100 of this embodiment, a plurality of metallic particles 30 is disposed on the light-transmissive layer 20. As described above, when the thickness G of the light-transmissive layer 20 is below approximately 40 nm, the mutual interaction between the localized surface plasmon in the vicinity of the metallic particles 30 and the propagating surface plasmon in the vicinity of the surface of the metallic layer 10 increases, and the ratio of the enhancement degree in the top of the metallic particles 30 to the enhancement degree in the bottom of the metallic particles 30 decreases. That is, the distribution of the energy for enhancing the electric field is biased to the bottom of the metallic particles 30.

It is considered that when the thickness G of the light-transmissive layer 20 is below approximately 40 nm, the electric field enhancement degree in the top of the metallic particles 30 with which the target substance is easily in contact relatively decreases even when a total electric field enhancement degree is not changed, and efficiency of enhancing the electric field of the electric field enhancing element 100 decreases. From such a viewpoint, according to the thickness G of the light-transmissive layer 20 which is set according to at least one of the conditions (i) and (ii), the ratio of the intensity of the localized surface plasmon (LSP) excited to the upper surface side (the top) of the metallic particles 30 to the intensity of the localized surface plasmon excited to the lower surface side (the bottom) of the metallic particles is constant regardless of the thickness G of the light-transmissive layer 20, and thus it is possible to increase usage efficiency of the energy of enhancing the electric field.

Furthermore, here, “constant” includes a case where a specific value does not vary, a case where the specific value varies in a range of ±10%, and preferably, a case where the specific value varies in a range of ±5%.

### 1.6. Excitation Light

The wavelength of the excitation light incident on the electric field enhancing element 100 generates the localized surface plasmon (LSP) in the vicinity of the metallic particles 30, and the wavelength of the excitation light is not limited insofar as at least one relationship of the conditions (i) and (ii) described in “1.2. Light-Transmissive Layer” is able to be satisfied, and is able to be an electromagnetic wave including ultraviolet ray, visible light, and infrared ray. The excitation light, for example, is able to be at least one of linearly polarized light polarized in the first direction, linearly polarized light polarized in the second direction, and circularly polarized light. According to this, it is possible to obtain an extremely great enhancement degree of light by the electric field enhancing element 100.

Furthermore, when the electric field enhancing element 100 is used as the SERS sensor, linearly polarized light polarized in the first direction, linearly polarized light polarized in the second direction, and circularly polarized light are suitably used in combination as the excitation light, and the number of enhancement degree peaks in the electric field enhancing spectrum, a size, and a shape (a width) may be adjusted to the wavelength of excitation light λᵢ, and the wavelength of Raman scattering light λₛ of the target substance.

The electric field enhancing element 100 of this embodiment has the following characteristics. The electric field enhancing element 100 of this embodiment is able to enhance light to an extremely high enhancement degree on the basis of plasmon excited by the light irradiation. The electric field enhancing element 100 of this embodiment has high enhancement degree, and thus for example, in a field such as medical treatment and health, environment, food, and public safety, a biologically-relevant substance such as a bacterium, a virus, a protein, a nucleic acid, and various antigens and antibodies, and various compounds including inorganic molecules, organic molecules, and high molecules are able to be used for a sensor for rapidly and simply performing detection with high sensitivity and high accuracy. For example, an antibody is bonded to the metallic particles 30 of the electric field enhancing element 100 of this embodiment, an enhancement degree at this time is obtained, and presence or absence of the antigen or an amount is able to be inquired on the basis of a change in a peak wavelength of an enhancement degree when a antigen is bonded to the antibody, or a change in reflectance of a wavelength which is set to the vicinity of the peak wavelength. In addition, by using the enhancement degree of the light in the electric field enhancing element 100 of this embodiment, it is possible to enhance the Raman scattering light of the trace substance.

### 2. ANALYSIS APPARATUS

An analysis apparatus of this embodiment includes the electric field enhancing element described above, a light source, and a detector. Hereinafter, a case where the analysis apparatus is a Raman spectroscopic device will be described as an example.

FIG. 10 is a diagram schematically illustrating a Raman spectroscopic device 200 according to this embodiment. The Raman spectroscopic device 200 detects and analyzes Raman scattering light from a target substance (qualitative analysis and quantitative analysis), and as illustrated in FIG. 7, includes a housing 140 containing a light source 210, a gaseous sample holding unit 110, a detection unit 120, a control unit 130, a detection unit 120, and a control unit 130. The gaseous sample holding unit 110 includes the electric field enhancing element according to the invention. Hereinafter, an example including the electric field enhancing element 100 described above will be described.

The gaseous sample holding unit 110 includes the electric field enhancing element 100, a cover 112 covering the electric field enhancing element 100, a suction flow path 114, and a discharge flow path 116. The detection unit 120 includes the light source 210, lenses 122a, 122b, 122c, and 122d, a half mirror 124, and a light detector 220. The control unit 130 includes a detection control unit 132 controlling the light detector 220 by processing a signal detected in the light detector 220, and an electric power control unit 134 controlling an electric power or a voltage of the light source 210 or the like. The control unit 130, as illustrated in FIG. 7, may be electrically connected to a connection unit 136 for being connected to the outside.

In the Raman spectroscopic device 200, when a suction mechanism 117 disposed in the discharge flow path 116 is operated, the inside of the suction flow path 114 and the discharge flow path 116 is negatively pressurized, and a gaseous sample including the target substance which is a detection target is suctioned from a suction port 113. A dust removing filter 115 is disposed in the suction port 113, and thus comparatively large dust, a part of water vapor, or the like is able to be removed. The gaseous sample is discharged from a discharge port 118 through the suction flow path 114 and the discharge flow path 116. When the gaseous sample passes through these paths, the gaseous sample is in contact with the metallic particles 30 of the electric field enhancing element 100.

The suction flow path 114 and the discharge flow path 116 have a shape in which light from the outside is not incident on the electric field enhancing element 100. Accordingly, light other than the Raman scattering light which is noise is not incident on the electric field enhancing element 100, and thus it is possible to improve an S/N ratio of the signal. A material configuring the flow paths 114 and 116, for example, is a material by which light is rarely reflected or a color.

The suction flow path 114 and the discharge flow path 116 have a shape in which fluid resistance with respect to the gaseous sample decreases. Accordingly, high sensitive detection is able to be performed. For example, the flow paths 114 and 116 have a smooth shape in which a corner portion is as fully eliminated as possible, and thus it is possible to prevent the gaseous sample from being accumulated in the corner portion. As the suction mechanism 117, for example, a fan motor or a pump of static pressure or air volume according to flow path resistance is used.

In the Raman spectroscopic device 200, the light source 210 irradiates the electric field enhancing element 100 with the excitation light. The light source 210 is arranged such that at least one of light linearly polarized in the first direction of the electric field enhancing element 100 (a direction in parallel with the metallic particles 30, and an extending direction of the metallic particle row 31) (linearly polarized light in the same direction as the first direction), light linearly polarized in the second direction, and circularly polarized light is able to be emitted. Though it is not illustrated, the incident angle θ of the excitation light emitted from the light source 210 may be suitably changed according to an excitation condition of the surface plasmon of the electric field enhancing element 100. The light source 210 may be disposed on a goniometer (not illustrated) or the like.

The light emitted by the light source 210 is identical to the light described in “1.6. Excitation Light”. Specifically, as the light source 210, a light source in which a wavelength select element, a filter, a polarizer, and the like are suitably disposed in a semiconductor laser, a gas laser, a halogen lamp, a high-pressure mercury lamp, a xenon lamp, and the like is able to be used as an example.

The light emitted from the light source 210 is focused on the lens 122a, and is incident on the electric field enhancing element 100 through the half mirror 124 and the lens 122b. SERS light is emitted from the electric field enhancing element 100, and the light reaches the light detector 220 through the lens 122b, the half mirror 124, and the lenses 122c and 122d. That is, the light detector 220 detects the light emitted from the electric field enhancing element 100. The SERS light includes Rayleigh scattering light having a wavelength identical to an incident wavelength from the light source 210, and thus the Rayleigh scattering light may be removed by a filter 126 of the light detector 220. The light from which the Rayleigh scattering light is removed is received by a light receiving element 128 as the Raman scattering light through a spectroscope 127 of the light detector 220. As the light receiving element 128, for example, a photodiode or the like is used.

The spectroscope 127 of the light detector 220, for example, is formed of an etalon or the like using a Fabry-Perot resonance, and is able to change a pass wavelength bandwidth. A Raman spectrum specific to the target substance is obtained by the light receiving element 128 of the light detector 220, and for example, the obtained Raman spectrum and data stored in advance are collated with each other, and thus it is possible to detect signal intensity of the target substance.

Furthermore, the Raman spectroscopic device 200 is not limited to the example described above insofar as the Raman spectroscopic device 200 includes the electric field enhancing element 100, the light source 210, and the light detector 220, the target substance is adsorbed by the electric field enhancing element 100, and the Raman scattering light is able to be acquired.

In addition, as in a Raman spectroscopic method according to this embodiment described above, when the Rayleigh scattering light is detected, the Raman spectroscopic device 200 may disperse the Rayleigh scattering light and the Raman scattering light by a spectroscope without having the filter 126.

The Raman spectroscopic device 200 includes the electric field enhancing element 100 described above. According to this Raman spectroscopic device 200 (an analysis apparatus), an extremely high enhancement degree is obtained in an enhancement degree (reflectance) spectrum, and it is possible to detect and analyze the target substance with high sensitivity. In addition, a position in which a high enhancement degree is obtained in the electric field enhancing element 100 provided in the Raman spectroscopic device 200 is positioned at least on the upper surface side (the top) of the metallic particles 30, and the target substance is easily in contact with the position, and thus it is possible to detect and analyze the target substance with high sensitivity.

In addition, this Raman spectroscopic device sets the thickness G of the light-transmissive layer 20 of the electric field enhancing element 100 according to at least one of the conditions (i) and (ii) described in “1.2. Light-Transmissive Layer”, and thus it is possible to increase an allowable range of a variation in manufacturing by setting the thickness G of the light-transmissive layer 20 to be greater than or equal to approximately 40 nm.

Further, according to this Raman spectroscopic device 200, the electric field enhancing element 100 in which a ratio of intensity of the localized surface plasmon excited to the lower surface side (the bottom) of the metallic particles 30 to intensity of the localized surface plasmon (LSP) excited to the upper surface side (the top) of the metallic particles is constant regardless of the thickness G of the light-transmissive layer 20 is used, and thus usage efficiency of energy of enhancing an electric field is high.

### 3. ELECTRONIC DEVICE

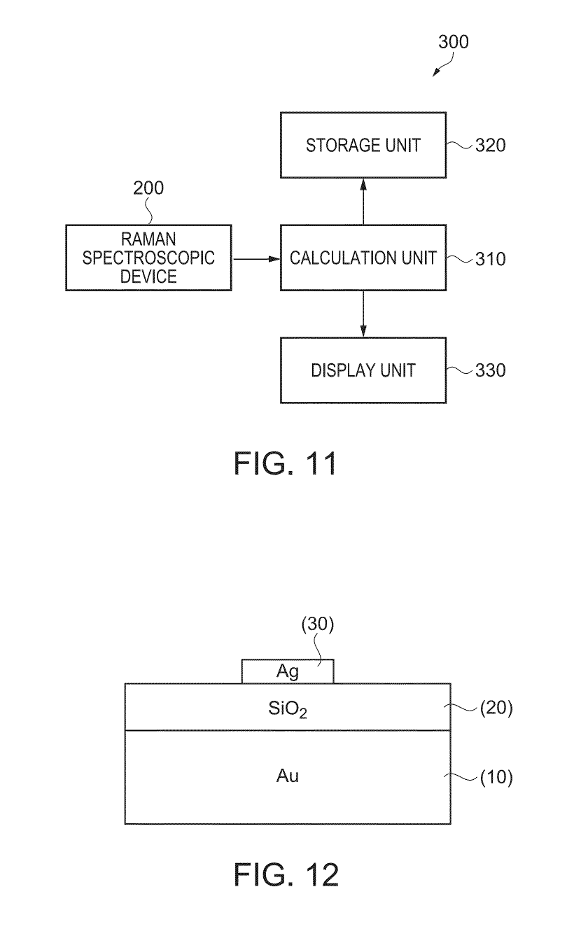

Next, an electronic device 300 according to this embodiment will be described with reference to the drawings. FIG. 11 is a diagram schematically illustrating the electronic device 300 according to this embodiment. The electronic device 300 is able to include the analysis apparatus (the Raman spectroscopic device) according to the invention. Hereinafter, as the analysis apparatus according to the invention, an example including the Raman spectroscopic device 200 described above will be described as an example.

The electronic device 300, as illustrated in FIG. 11, includes the Raman spectroscopic device 200, a calculation unit 310 which calculates medical health information on the basis of detection information from the light detector 220, a storage unit 320 which stores the medical health information, and a display unit 330 which displays the medical health information.

The calculation unit 310, for example, is a personal computer or a personal digital assistant (PDA), and receives detection information (a signal or the like) transmitted from the light detector 220. The calculation unit 310 calculates the medical health information on the basis of the detection information from the light detector 220. The calculated medical health information is stored in the storage unit 320.

The storage unit 320, for example, is semiconductor memory, a hard disk drive, or the like, and may be configured to be integrated with the calculation unit 310. The medical health information stored in the storage unit 320 is transmitted to the display unit 330.

The display unit 330, for example, is configured by a display plate (a liquid crystal monitor or the like), a printer, an illuminator, a speaker, and the like. The display unit 330 displays or activates an alarm on the basis of the medical health information or the like calculated by the calculation unit 310 such that a user is able to recognize contents thereof.

As the medical health information, information relevant to presence or absence or an amount of at least one biologically-relevant substance selected from a group consisting of a bacterium, a virus, a protein, a nucleic acid, and an antigen and antibody, or at least one compound selected from inorganic molecules and organic molecules is able to be included.

The electronic device 300 includes the Raman spectroscopic device 200 described above. For this reason, in the electronic device 300, detection of a trace substance is able to be more efficiency performed with high sensitivity, and it is possible to provide medical health information with high accuracy.

For example, the electric field enhancing element according to the invention is able to be used as an affinity sensor or the like which detects presence or absence of adsorption of a substance such as presence or absence of adsorption of an antigen in an antigen-antibody reaction. In the affinity sensor, white light is incident on the sensor, a wavelength spectrum is measured by a spectroscope, and a shift amount of a surface plasmon resonance wavelength due to adsorption is detected, and thus adsorption of a detection substance with respect to a sensor chip is able to be detected with high sensitivity.

### 4. EXPERIMENTAL EXAMPLE

Hereinafter, the invention will be further described by using experimental examples, but the invention is not limited to the following examples.

In each experimental example, the following model schematically illustrated in FIG. 12 is used.

As a metallic layer which is sufficiently thick to the extent that light is not transmitted, a gold (Au) layer is used, as a light-transmissive layer, a SiO₂ layer having a refractive index of 1.46 is formed on the metallic layer (gold), and as metallic particles, cylindrical silver is formed on the light transmissive layer at a constant cycle, and thus a Gap type Surface Plasmon Polariton (GSPP) model is formed. Furthermore, a material of the metallic layer and the metallic particles is not limited, insofar as metal in which a real part of a dielectric constant negatively increases, and an imaginary part is smaller than the real part in a wavelength region of the excitation light is used, plasmon is able to be generated.

Parameter or the like of Calculation Model

In a graph or the like illustrated as each experimental example, for example, a signage such as “X780Y780” is used. “X780Y780” indicates that metallic particles are arranged in the first direction (an X direction) at a pitch of 780 nm (the first pitch P1) and in the second direction (a Y direction) at a pitch of 780 nm (the second pitch P2).

In addition, when a character such as “D” and “T” is applied to a numerical value, it indicates that the metallic particles used in the model are in the shape of a cylinder having a diameter D and a height T. In addition, when a symbol “G” is further applied to the numerical value, it indicates that the thickness G of the light-transmissive layer is the numerical value [nm] described above. In addition, a Gap thickness in the horizontal axis of the graph indicates the thickness G of the light-transmissive layer. Further, when the numerical value, for example, is written with a range such as “20 to 100”, it indicates that calculation is performed by adopting a continuous or infrequent (discrete) value as the numerical value described above on calculation in the range described above.

Further, “Ag” or “AG” in the drawings indicates that a material of a configuration of focus is silver, and “Au” or “AU” indicates that a material of a configuration of focus is gold. In addition, “@” indicates “in a wavelength followed by @”, and for example, “SQRT_{—}@815 nm” indicates SQRT in a wavelength of 815 nm.

Furthermore, in the model, SiO₂ is formed on the metallic layer of gold as the light-transmissive layer, silver or gold is formed at a predetermined pitch as the metallic particles, and as the diameter of the metallic particles, a size in which a mutual interaction between LSP and PSP increases is selected. Except Experimental Example 8, the pitch is a pitch of 780 nm and a pitch of 600 nm corresponding to an excitation wavelength of 785 nm and 633 nm in a vertical incidence.

### Outline of Calculation

The calculation is performed by using FDTD soft FullWAVE manufactured by Rsoft (currently, Cybernet Systems Co., Ltd.). In addition, a condition of the used mesh will be described in each experimental example, and for example, “XY1Z1-5nmGG” indicates “XY1nmZ1-5nm Grid Grading”, and “2-10nmGG” indicates “XYZ2-10nm Grid Grading”. In addition, a calculation time cT is 10 μm.

In addition, the peripheral refractive index n₀ of the metallic particles is 1. In all of the experimental examples, the material of the light-transmissive layer is SiO₂. In addition, the excitation light is in a vertical incidence from the thickness direction (Z) of the light-transmissive layer, and is linearly polarized light in the X direction.

In each experimental example, near-field properties and/or far-field properties are obtained. As an FDTD calculation condition of the near-field properties, a 1 nm mesh even in XY directions, a grid grating (GG) of 1 nm to 5 nm in a Z direction (calculation time cT=10 μm), or GG of 2 nm to 10 nm in XYZ directions (calculation time cT=7 μm) is used. In addition, a condition of the used mesh will be described in each experimental example, and for example, “XY1Z1-5nmGG” indicates “XY1nmZ1-5nm Grid Grading”, and “2-10nmGG” indicates “XYZ2-10nm Grid Grading”.

In an enhancing position (a hot spot), two components of electric fields Eₓ and E_{z} are formed, and thus an entire enhancement degree in the following experimental examples is expressed by SQRT (Eₓ²+E_{z}²). Here, Eₓ represents intensity of an electric field in a polarization direction (the first direction) of incident light, and E_{z} indicates electric field intensity in the thickness direction. Furthermore, in this case, the electric field intensity in the second direction is small, and thus it is not considered. In addition, hereinafter, SQRT (Eₓ²+E_{z}²) is simply referred to as “SQRT”.

In addition, when the surface plasmon resonance (SPR) is generated due to the irradiation of the excitation light, absorption occurs due to the resonance, and thus reflectance decreases. For this reason, intensity in an SPR enhanced electric field is able to be expressed by (1−r) using reflectance r. According to a relationship in which intensity in an enhanced electric field is strong as a value of the reflectance r becomes closer to zero, the reflectance is used as an index of the square of the intensity (SQRT) in the SPR enhanced electric field.

In an FDTD calculation condition of the far-field properties, a monitor is disposed away from an element, pulse light having a center wavelength of 0.5 μm is incident as the excitation light, and a wavelength characteristic of the reflectance is acquired. According to this method, a minimum value of the reflectance indicates a greatest value of an enhancement degree, and a wavelength having a peak at which an enhancement degree is maximized is also able to be acquired. In addition, the far-field properties are an integration value of the near-field properties in a hot spot of each portion, and in general, a result which is approximately identical to that of the near-field properties is able to be obtained. The far-field properties are mainly acquired at 2 nmGG to 10 nmGG, and a calculation time cT is 32.7 μm.

Furthermore, in the far-field properties, when an abnormal value depending on a mesh size occurs, the mesh size is set to 1 nmGG to 5 nmGG, and the calculation is performed again.

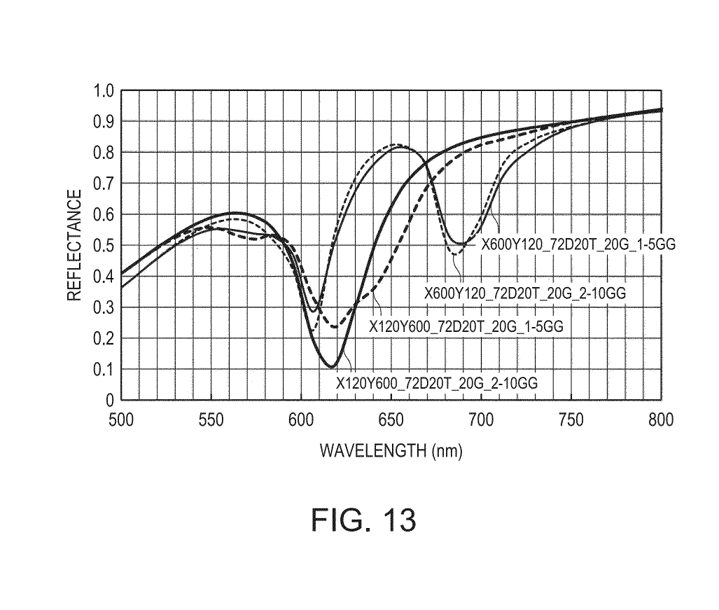

In FIG. 13, an example of the far-field properties (a reflectance spectrum) calculated by changing the mesh size with respect to a specific model is illustrated.

It is found that a peak value of a peak in the reflectance spectrum and a reflectance minimum value are approximately identical to each other in the mesh size of 1 nmGG to 5 nmGG and 2 nmGG to 10 nmGG. Here, a decrease in reflectance is approximately identical to an increase in a plasmon enhancement degree.

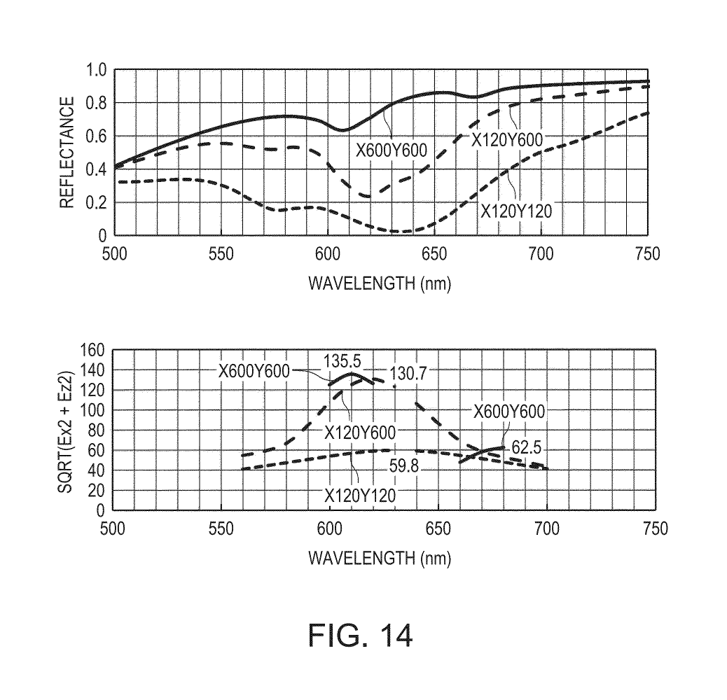

Next, in the specific model, spectrums of the far-field properties and the near-field properties are compared (FIG. 14).

From FIG. 14, according to this model, it is found that wavelengths having peaks appearing in the far-field properties and the near-field properties approximately coincident with each other. However, sizes of the wavelengths having the peaks appearing in far-field properties and the near-field properties between models which are different from each other are not necessarily coincident with each other. This is because densities of arranging the metallic particles on the light-transmissive layer are different from each other.

### 4.1. Experimental Example 1

It is difficult to completely exclude a variation in a size of the metallic particles of the electric field enhancing element in manufacturing the element. The inventors have prepared and analyzed a plurality of electric field enhancing elements including metallic particles having a diameter of 150 nm by using an electron beam drawing device (EB), and have found that a distribution (a variation) of a standard deviation σ=5 nm occurs in the diameter of the metallic particles. That is, it has been found that as a premise of this experimental example, that is, as the diameter of the metallic particles, a difference between the greatest diameter and the smallest diameter in average is approximately 10 nm.

Therefore, in this experimental example, due to a resonance of the localized surface plasmon (LSP) and the propagating surface plasmon (PSP), an influence of a variation in a size of the metallic particles on a peak of an enhancement degree (reflectance) spectrum is inquired by a simulation of the calculator using a model exhibiting anti-crossing behavior.

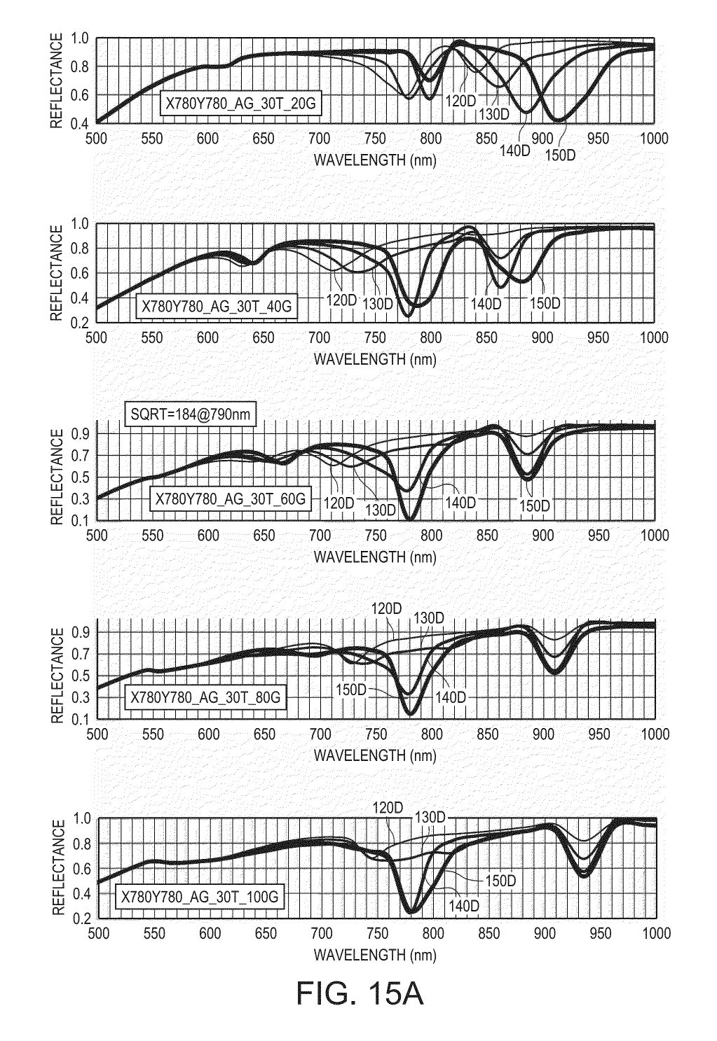

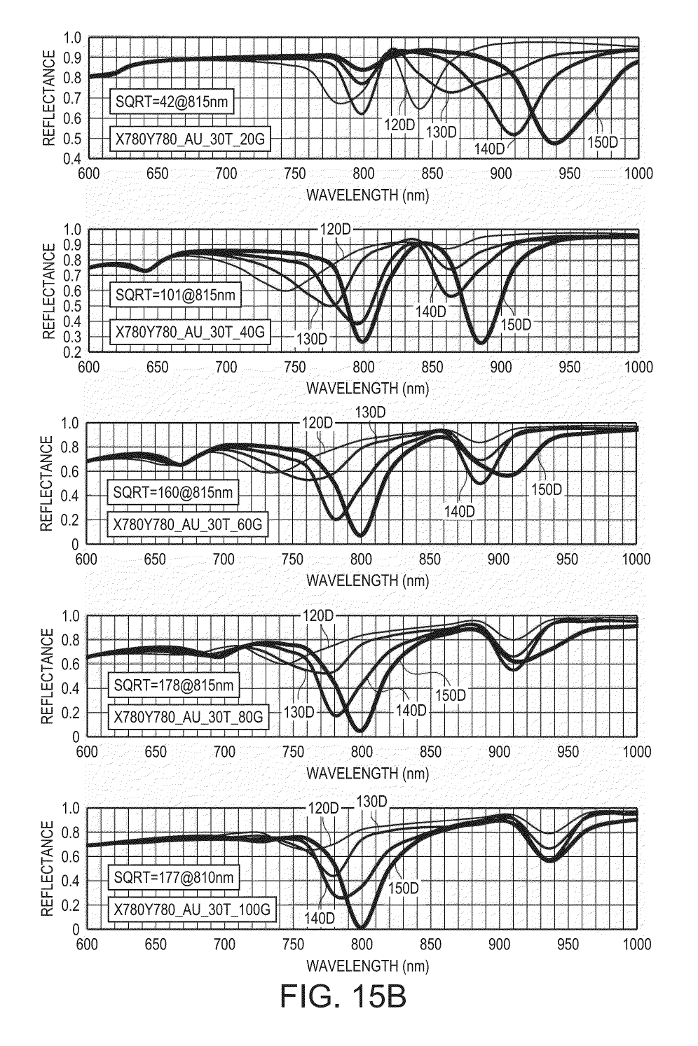

FIG. 15A illustrates a calculation result of X780Y780_{—}120-140D30T_AG (a silver particle model (a))_{—}20-100G, and FIG. 15B illustrates a calculation result of X780Y780_{—}130-150D30T_AU (a gold particle model (b))_{—}20-100G.

From the calculation result of the silver particle model illustrated in FIG. 15A, in 20G (the thickness of the light-transmissive layer is 20 nm), it is found that a peak appearing on a short wavelength side in a reflectance spectrum is shifted by 12.5 nm, and a peak appearing on a long wavelength side is shifted by 22.5 nm by changing the diameter of the silver particles by 10 nm. In addition, from the calculation result of the silver particle model illustrated in FIG. 15B, in 20G, it is found that the peak appearing on the short wavelength side in the reflectance spectrum is not shifted, but the peak appearing on the long wavelength side is shifted by 37.5 nm.

On the other hand, as illustrated in FIGS. 15A and 15B, in 100G (the thickness of the light-transmissive layer is 100 nm), it is found that the peak appearing on the short wavelength side in the reflectance spectrum is shifted by approximately 15 nm and the peak appearing on the long wavelength side is not shifted in the silver particle model, and the peak appearing on the short wavelength side in the reflectance spectrum is shifted by approximately 10 nm and the peak appearing on the long wavelength side is not shifted in the gold particle model.

In addition, from the results of FIGS. 15A and 15B, in 60G at the time of using the silver particles and in 100G at the time of using gold particles, it is suggested that there is a condition in which a smallest value of reflectance of the peak on the short wavelength side greatly decreases (an enhancement degree of plasmon increases), and the peak on the long wavelength side is rarely shifted.

From the result of this experimental example, in a case where the thickness G of the light-transmissive layer is 20 nm, it is found that when the diameter D of the metallic particles is changed by approximately 10 nm (that is, when a variation occurs in a particle diameter of the metallic particles in the electric field enhancing element), a peak appearing in a reflectance (an enhancement degree) profile (a reflectance spectrum) (a spectrum indicating a change in reflectance (an enhancement degree) with respect to a wavelength) of the electric field enhancing element greatly varies at least in a position.

### 4.2. Experimental Example 2

Similar to Experimental Example 1, in a model of this experimental example, SiO₂ is formed on the metallic layer of gold as the light-transmissive layer, and silver or gold is formed at a predetermined pitch as the metallic particles. The diameter of the metallic particles is in a size where a mutual interaction between LSP and PSP increases. The pitch is a pitch of 780 nm and a pitch of 600 nm corresponding to an excitation wavelength of 785 nm and 633 nm.

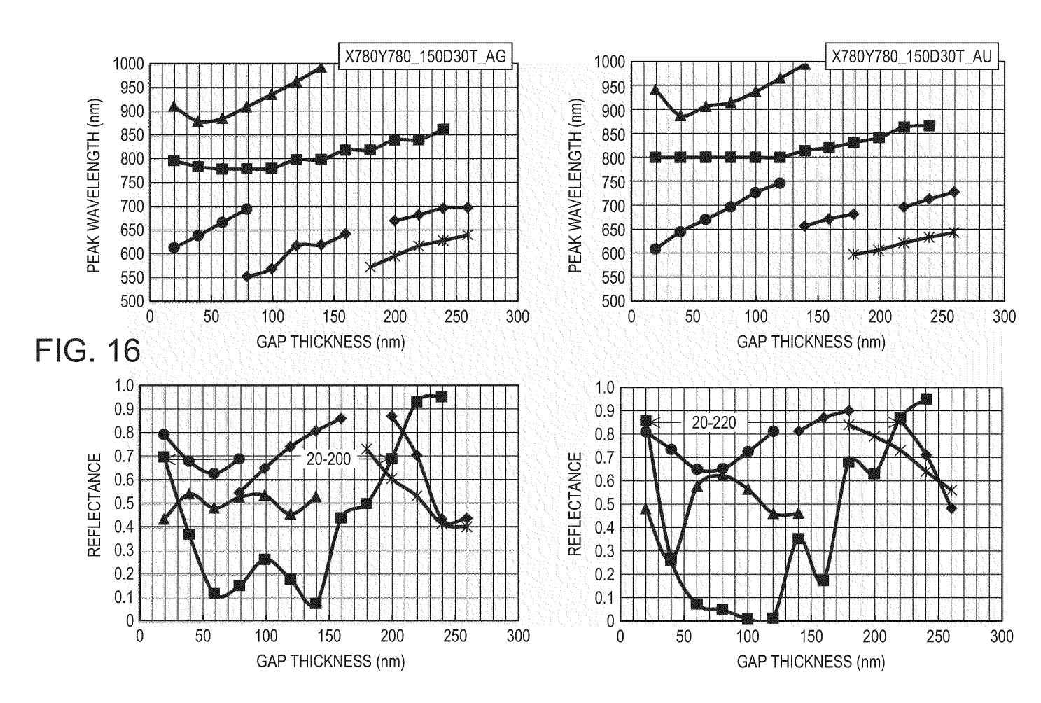

FIG. 16 illustrates dependent properties of a wavelength having a peak in a reflectance spectrum of a model of X780Y780_{—}150D30T_AG and X780Y780_{—}150D30T_AU (an upper portion in the drawing), and a minimum value of the peak in the reflectance spectrum (indicating a peak top value in a downward peak) (a lower portion in the drawing) with respect to the thickness G of the light-transmissive layer. The diameter D of the metallic particles in this model is 150D by selecting a value at which the enhancement degree increases most.

In this model, it is found that each peak on the short wavelength side (a black square (a filled square) in the drawing) in G=40 nm to 200 nm of the silver particles and in G=40 nm to 220 nm of the gold particles is smaller than the reflectance in G=20 nm (the enhancement degree increases). It is found that a value of the reflectance corresponding to the peak on the long wavelength side (a black triangle (a filled triangle) in the drawing) is rarely changed even when G increases from a value of G=20 nm. In addition, when as the thickness G of the light-transmissive layer at which an enhancing effect due to an interference effect is dominant in this model, a thickness at which the reflectance is 0.4 to 0.6 or less is read from FIG. 16, the thickness G is approximately 240 nm in the silver particles and is greater than or equal to approximately 260 nm in the gold particles, and en effect that the thickness G of the light-transmissive layer of the silver particles has a relationship of 40 nm≦G≦200 nm and the thickness G of the light-transmissive layer of the gold particles has a relationship of 40 nm≦G≦220 nm does not correspond to an interference resonance effect.

Next, in 60G of the silver particles and 100G of the gold particles in which the reflectance minimum value decreases most, the near-field properties are calculated. A mesh used for this calculation is XY1Z1-5nmGG, and cT is 10 μm.

As a result thereof, it is found that in X780Y780_{—}150D30T_AG_{—}60G, SQRT in a bottom of the silver particles is SQRT=184@790 nm and SQRT=93@890 nm, and in X780Y780_{—}150D30T_AU_{—}100G, SQRT in a bottom of the gold particles is SQRT=177@810 nm and SQRT=80@960 nm, and thus an extremely high enhancement degree is obtained. That is, it is found that the near-field properties are acquired at a dimension where small reflectance is obtained in the far-field properties, and extremely high SQRT is obtained, and thus the far-field properties and the near-field properties preferably correlate with each other.

Next, in the model of X780Y780_{—}150D30T_AU, dependent properties of the near-field properties with respect to the thickness G of the light-transmissive layer are calculated. A mesh used in this calculation is XY1Z1-5nmGG, and cT is 10 μm. In addition, in this calculation, the excitation wavelength is fixed to 815 nm.

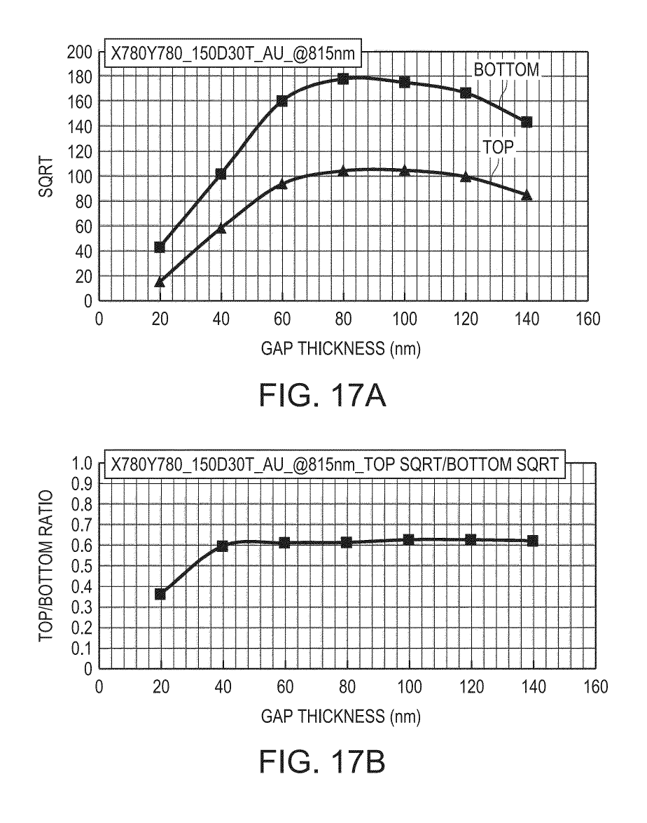

FIG. 17A is a graph of dependent properties of SQRT@815 nm of the model of X780Y780_{—}150D30T_AU with respect to the thickness G of the light-transmissive layer. FIG. 17B is a graph of dependent properties of a top SQRT/bottom SQRT ratio (a ratio of intensity of the localized surface plasmon excited to the upper surface side of the metallic particles to intensity of the localized surface plasmon excited to the lower surface side of the metallic particles) with respect to the thickness G of the light-transmissive layer. FIGS. 17A and 17B correspond to FIG. 15B in that SQRT of a near-field in the peak on the short wavelength side of Au is inquired by fixing the excitation wavelength to 815 nm.

From FIG. 17A, it is found that in the top and the bottom of the metallic particles, a SQRT value indicates dependence properties of the thickness G of the light-transmissive layer which are similar to each other. In addition, from FIG. 17B, it is found that the top SQRT/bottom SQRT ratio is an approximately constant value (in this example, approximately 0.6) when the thickness G of the light-transmissive layer is greater than or equal to 40 nm.

Further, in FIG. 17A, when the thickness G of the light-transmissive layer is 20 nm, SQRT is a small value in the top and the bottom. It is considered that this is because the peak on the short wavelength side when the thickness G is 20 nm (a resonance wavelength) is greatly shifted from 815 nm to the long wavelength side.

As described above, in this experimental example, the following is found. It is found that when the thickness G of the light-transmissive layer is less than 40 nm, the top SQRT/bottom SQRT ratio decreases without depending on a model. In contrast, it is found that when the thickness G of the light-transmissive layer is greater than or equal to 40 nm, the top SQRT/bottom SQRT ratio is approximately constant without depending on a model. That is, it is found that when the thickness G of the light-transmissive layer is less than 40 nm, the electric field enhancement degree in the top of the metallic particles with which the target substance is easily in contact relatively decreases, and when the thickness G of the light-transmissive layer is greater than or equal to 40 nm, a ratio of the intensity of LSP excited to the top of the metallic particles to the intensity of LSP excited to the bottom of the metallic particles is constant regardless of the thickness G of the light-transmissive layer.

In addition, from this experimental example, it is found that the thickness G of the light-transmissive layer is set to be thick, and thus the intensity of LSP in the thickness direction decreases. On the other hand, it is found that the thickness G of the light-transmissive layer is set to be thick, and thus the intensity of PSP occurring in the X direction and the Y direction increases. LSP strongly occurs in the polarization direction of the excitation light, but PSP does not influence on the polarization direction of the excitation light, and as illustrated in FIG. 9, PSP strongly occurs by a diffraction grating passing through the intersecting point of the dispersion relationship. Here, FIG. 9 is a case where the excitation light is in the vertical incidence, and when a diffraction grating pitch Q completing the expression (C) described above is completed in the oblique incidence, PSP strongly occurs in this direction. As described above, it is found that the model of this experimental example is a mode based on PSP because PSP occurs in the X direction and the Y direction, and dependent properties of PSP with respect to the thickness G of the light-transmissive layer are strongly obtained.

### 4.3. Experimental Example 3

Similar to Experimental Example 1, in a model of this experimental example, SiO₂ is formed on the metallic layer of gold as the light-transmissive layer, and silver or gold is formed at a predetermined pitch as the metallic particles. The diameter of the metallic particles is in a size where a mutual interaction between LSP and PSP increases. The pitch is a pitch of 780 nm and a pitch of 600 nm corresponding to an excitation wavelength of 785 nm and 633 nm.

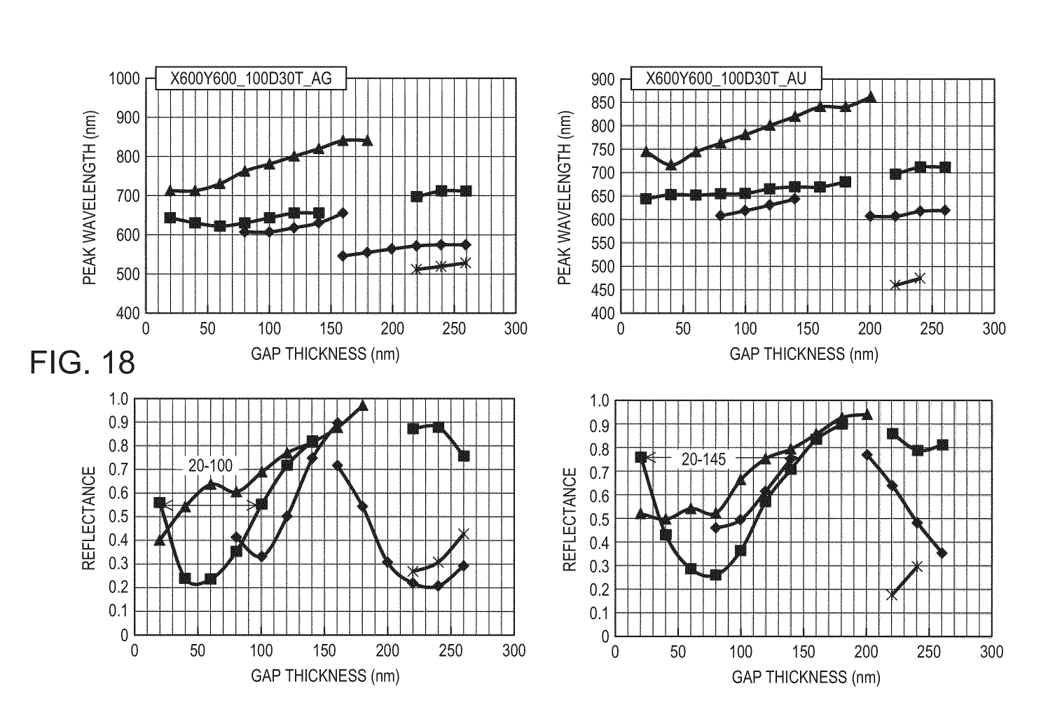

FIG. 18 illustrates dependent properties of a wavelength having a peak in a reflectance spectrum of a model of X600Y600_{—}100D30T_AG and X600Y600_{—}100D30T_AU, and a minimum value of the peak in the reflectance spectrum with respect to the thickness G of the light-transmissive layer. Gap thickness dependent properties of the wavelength having a peak and the minimum value of the reflectance are obtained from a reflectance spectrum in a far-field, and a mesh is XYZ2-10GG. FIG. 18 is a graph in which a peak wavelength and a reflectance minimum value are plotted with respect to the thickness G of the light-transmissive layer for each model. The diameter D of the metallic particles in this model is 100D by selecting a value at which the enhancement degree increases most.

From FIG. 18, a value of G which is below the reflectance in 20G (an enhancement degree is high) is as follows. The value of G in X600Y600_{—}100D30T_AG is 20 nm to 100 nm, and the value of G in X600Y600_{—}100D30T_AU is 20 nm to 145 nm.

On the other hand, from FIG. 16 described in Experimental Example 2, the value of G which is below the reflectance in 20G (the enhancement degree is high) is as follows. The value of G in X780Y780_{—}150D30T_AG is 20 nm to 200 nm, and the value of G in X780Y780_{—}150D30T_AG is 20 nm to 220 nm.

Here, the obtained reflectance is a value of the top and the bottom of the metallic particles, or an integration value of values in other hot spots. For this reason, in the following Experimental Example 4, an enhancement degree in the top of the metallic particles which is an advantageous portion for sensing is inquired.

### 4.4. Experimental Example 4

In this experimental example, dependent properties of an enhancement degree in a hot spot with respect to the thickness G of the light-transmissive layer are inquired. With respect to the result of the far-field in Experimental Example 3 described above, the near-field properties in the top of the metallic particles which are an important hot spot as a sensing portion are acquired. The used mesh is 2GG to 10 GG. FIG. 19 shows graphs illustrating thickness dependent properties of SQRT in the top of the metallic particles when the diameter D of the metallic particles of each model is changed with respect to the light-transmissive layer.

From FIG. 19, when the diameter D of the metallic particles is changed, light-transmissive layer thickness dependent properties of SQRT are changed. This is because when the diameter of the metallic particles increases, the peak wavelength of LSP is shifted to the long wavelength side, and when the diameter of the metallic particles decreases, the peak wavelength of LSP is shifted to the short wavelength side, and thus a mutual interaction between LSP and PSP is changed in a fixed wavelength (each excitation wavelength). It is able to be considered that each excitation wavelength is fixed to 785 nm and 633 nm, and thus a line indicating the highest SQRT is the diameter of the metallic particles at which LSP and PSP are preferably matched to each other (the mutual interaction increases).

Then, from FIG. 19, the value of G in which the hot spot in the top of the metallic particles exceeds SQRT of 20G is as follows. The value of G in X600Y600_AG@633 nm is 20 nm to 125 nm, the value of G in X600Y600_AU@633 nm is 20 nm to 120 nm, the value of G in X780Y780_AG@785 nm is 20 nm to 145 nm, and the value of G in X780Y780_AU@785 nm is 20 nm to 140 nm.

In addition, from this result, it is found that a range of G is not greatly changed in the silver particles and the gold particles, and the enhancement degree increases in a range of 20 nmG to 120 nmG in an excitation model of 633 nm and in a range of 20 nmG to 140 nmG in an excitation model of 785 nm.

### 4.5. Experimental Example 5

As Experimental Example 5, results of Experimental Example 1 to Experimental Example 4 described above are summarized. Thus, the following is qualitatively confirmed.

From Experimental Example 1 and Experimental Example 2, it is found that in a range of 20 nm≦G<40 nm, the mode is on the basis of LSP in the thickness direction of the light-transmissive layer and between the metallic particles, a plasmon enhancing peak wavelength with respect to a variation in the diameter of the metallic particles is greatly shifted, and a top and bottom ratio of the metallic particles varies.

In addition, from Experimental Examples 2 to 4, it is found that in a range of 40 nm≦G, both of the top and the bottom of the metallic particles are a mode based on a product of LSP and PSP in the thickness direction, a plasmon enhancing peak wavelength shift with respect to the variation in the diameter of the metallic particles decreases, and the top and bottom ratio of the metallic particles is constant.

Then, from Experimental Example 2, the mode is on the basis of the interference effect in the thickness direction from a portion at which the value of G exceeds 200 nm and has a small effect of LSP between the metallic particles. In addition, with respect to the variation in the diameter of the metallic particles, a wavelength shift in a peak is small, but it is difficult to change the value of SQRT to be sensitive to the value of G and to expect a high enhancement degree in a wide wavelength range due to a sharp reflectance spectrum.

### 4.6. Experimental Example 6

In this experimental example, on the basis of results of each experimental example described above, a preferred parameter of the electric field enhancing element according to the invention is derived.

From FIG. 19, in X780Y780 of the excitation model of 785 nm and X600Y600 of the excitation model of 633 nm, G indicating SQRT exceeding SQRT of 20 nmG is 20 nm to 140 nm in the excitation model of 785 nm, and is 20 nm to 120 nm in the excitation model of 633 nm. A preferred value of G is changed by the excitation wavelength.

Accordingly, the following expression is derived.

20 nm≦G≦140 nm·excitation wavelength/785 nm

Here, this range is a range of G derived from a case where the material of the light-transmissive layer is SiO₂ having n=1.46 in the vertical incidence.

The thickness G of the light-transmissive layer in a structure of each experimental example is shifted according to the reflective index of the used light-transmissive layer with respect to the range of G when SiO₂ is used as a base. Specifically, when a preferred range is 20 nm to 140 nm in SiO₂, the thickness of the light-transmissive layer when TiO₂ having a reflective index of 2.49 is used for the light-transmissive layer is obtained by multiplying the thickness in SiO₂ by (1.46/2.49), and a preferred range of the thickness in TiO₂ is 12 nm to 82 nm.

In addition, the light-transmissive layer may be formed of a multi-layer. For example, Al₂O₃ having a reflective index of 1.64 is formed on the metallic layer side of the light-transmissive layer to be 10 nm as an adhesive layer, and when SiO₂ is formed thereon to be 30 nm, the same effect as that of SiO₂ of (1.64·10+1.46·30)/1.46=41.2 nm is obtained by using an arithmetic average (that is, an effective reflective index) of each layer with respect to the reflective index.

In addition, in order to be generalized to a case other than the vertical incidence, a method of considering a geometric light path length, and a method of considering an incident angle of the excitation light with respect to the light-transmissive layer and diffraction inside the light-transmissive layer are considered. Then, in consideration of results of Experimental Example 1 and Experimental Example 2 described above, when a lower limit value of G is 20 nm, a range as described in “1.2. Light-Transmissive Layer” is derived.

### 4.7. Experimental Example 7

A model in which SiO₂ is formed on the metallic layer of gold as the light-transmissive layer, and silver or gold is formed at a predetermined pitch as the metallic particles is simulated. The diameter of the metallic particles is in a size where a mutual interaction between LSP and PSP increases. The pitch is a pitch of 780 nm and a pitch of 600 nm corresponding to an excitation wavelength of 785 nm and 633 nm.

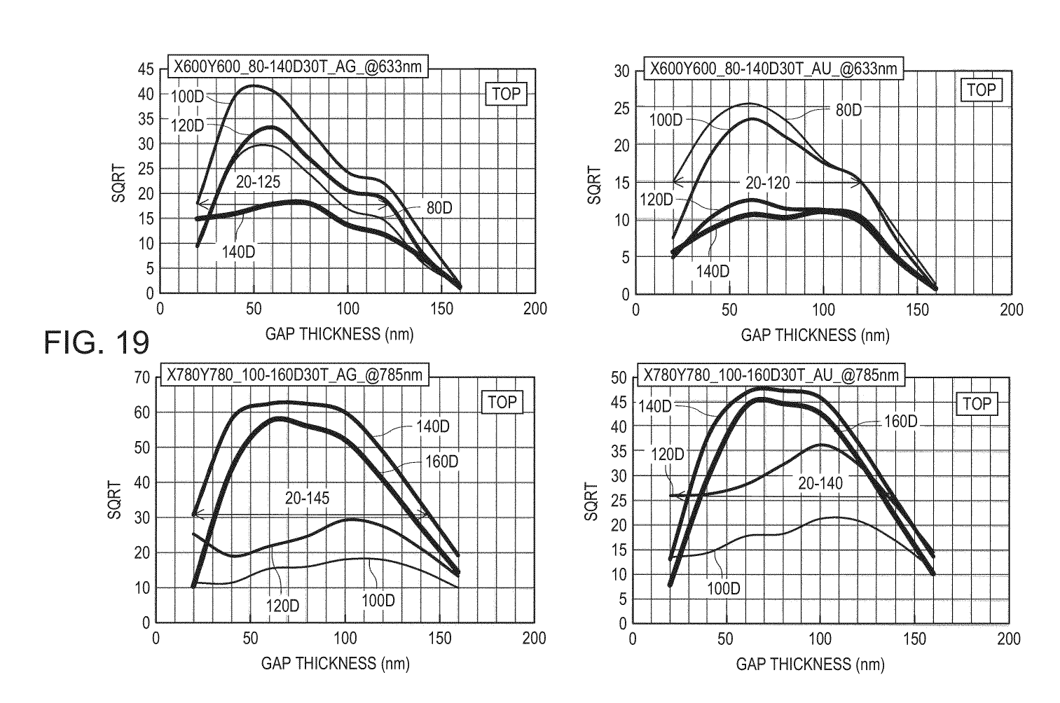

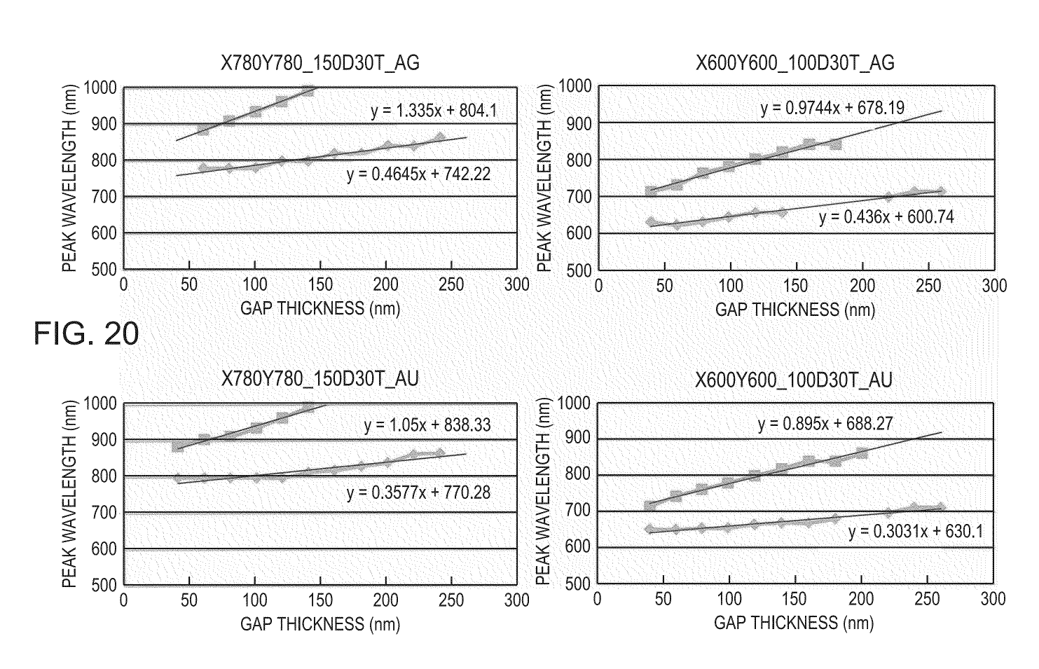

FIG. 20 shows graphs illustrating dependent properties of a peak wavelength in a reflectance spectrum of this model with respect to the thickness G of the light-transmissive layer. From FIG. 20, it is found that in the thickness of SiO₂ (the thickness G of the light-transmissive layer) is in a range of 40 nm to 140 nm in any model, a peak wavelength having a peak on the short wavelength side (a black rhombus (a black diamond) (a filled rhombus; a filled diamond)) is rarely changed, and a peak wavelength having a peak on the long wavelength side (a black square (a filled square)) is shifted to the long wavelength side as the thickness of SiO₂ becomes thicker.

It is found that as the Raman spectroscopic device (the analysis apparatus), a SERS sensor having high enhancing effect with respect to both of the excitation light and the Raman scattering light is able to be provided by adopting a structure of this experimental example as the electric field enhancing element, and by designing the thickness G of the light-transmissive layer such that a wavelength having a peak of an enhancement degree corresponds to the wavelength of the Raman scattering light or the wavelength of the excitation light of the target substance using this phenomenon. For example, in the excitation model of 633 nm, when G is 40 nm, a peak in the vicinity of 710 nm on the long wavelength side is linearly shifted from 710 nm to 813 nm as G becomes greater, and in the excitation model of 785 nm, when G is 40 nm, a peak in the vicinity of 880 nm on the long wavelength side is linearly shifted from 880 nm to 976 nm as G becomes greater. For this reason, by setting the enhancement degree using this peak, it is possible to adjust SERS measurement to be performed with high sensitivity with respect to the target substance of which a value of the Raman shift is in a range of 1750 cm⁻¹ to 3500 cm⁻¹ in the model of 633 nm and in a range of 1400 cm⁻¹ to 2500 cm⁻¹ in the excitation model of 785 nm. Then, the peak in the vicinity of the wavelength of the excitation light is not greatly changed even when the value of G is changed, and thus it is possible to maintain the enhancement degree in the wavelength of the excitation light to be great and to change the value of G such that the enhancement degree in the wavelength of the Raman scattering light increases, and it is possible to extremely easily design the value of G.

Further, specifically, when the target substance is acetone, a wavenumber (a Raman shift) of the stokes Raman scattering light is 787 cm⁻¹, 1708 cm⁻¹, and 2921 cm⁻¹. Then, when the wavelength of excitation light λᵢ is 633 nm, each wavelength λₛ of stokes Raman scattering light is 666 nm, 709 nm, and 777 nm corresponding to the Raman shift of acetone.

Similarly, when the wavelength of excitation light λᵢ is 785 nm, each wavelength of stokes Raman scattering light λₛ is 837 nm, 907 nm, and 1019 nm corresponding to the Raman shift of acetone.

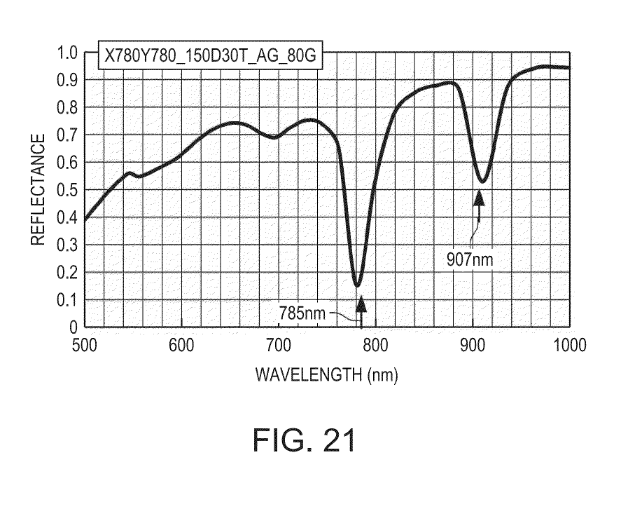

Here, FIG. 21 is a graph illustrating the wavelength characteristic of the enhancement degree of the electric field enhancing element, and the excitation wavelength and the scattering wavelength of SERS. As illustrated in FIG. 21, in order to detect the Raman shift of 1708 cm⁻¹ of acetone, the excitation wavelength λᵢ is 785 nm, and the wavelength of stokes Raman scattering light λₛ is 907 nm, and thus X780Y780_{—}150D30T_{—}80G_AG may be used, and according to this, it is possible to obtain a strong SERS signal in the Raman shift of 1708 cm⁻¹ of acetone.

### 4.8. Experimental Example 8

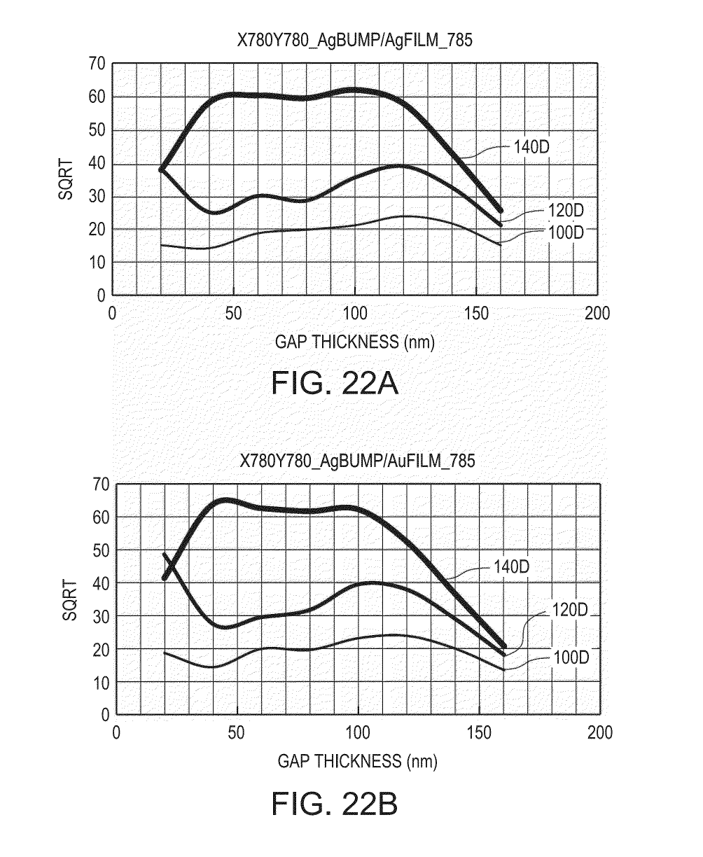

Experimental Examples 1 to 7 described above are calculated by using gold as the material of the metallic layer. In this experimental example, the material of the metallic layer is changed to silver, and dependent properties of SQRT with respect to the thickness G of the light-transmissive layer are inquired. FIG. 22A is a graph illustrating dependent properties of SQRT of X780Y780_{—}100-140D30T_AG (silver particles)@785 nm with respect to the thickness G of the light-transmissive layer when the material of the metallic layer is silver, and FIG. 22B is a graph illustrating dependent properties of SQRT of X780Y780_{—}100-140D30T_AG (silver particles)@785 nm with respect to the thickness G of the light-transmissive layer when the material of metallic layer is gold. Furthermore, a mesh of 2 GG to 10 GG is used.

From FIGS. 22A and 22B, it is found that in both of the case where the material of the metallic layer (the mirror layer) is silver and the case where the material of the metallic layer is gold, there is no great difference in the dependent properties of SQRT with respect to the thickness G of the light-transmissive layer.

In addition, in Experimental Examples 1 to 7 described above, SiO₂ is used as the material of the light-transmissive layer, and Al₂O₃, TiO₂, and the like may be used. When a material other than SiO₂ is used, the thickness G of the light-transmissive layer may be set in consideration of a reflective index of the material other than SiO₂ by using SiO₂ of Experimental Examples 1 to 7 described above as a base. For example, in a case where it is preferable that the thickness of the light-transmissive layer when the material is SiO₂ is in a range greater than 20 nm and less than or equal to 140 nm, when the material of the light-transmissive layer is TiO₂, a preferred thickness G of the light-transmissive layer is able to be obtained by multiplying the thickness of the light-transmissive layer when the material is SiO₂ by a value of (1.46/2.49) in consideration of a refractive index (2.49) of TiO₂. Therefore, when the material of the light-transmissive layer is TiO₂, the preferred thickness G of the light-transmissive layer is approximately greater than 12 nm and less than or equal to 82 nm.

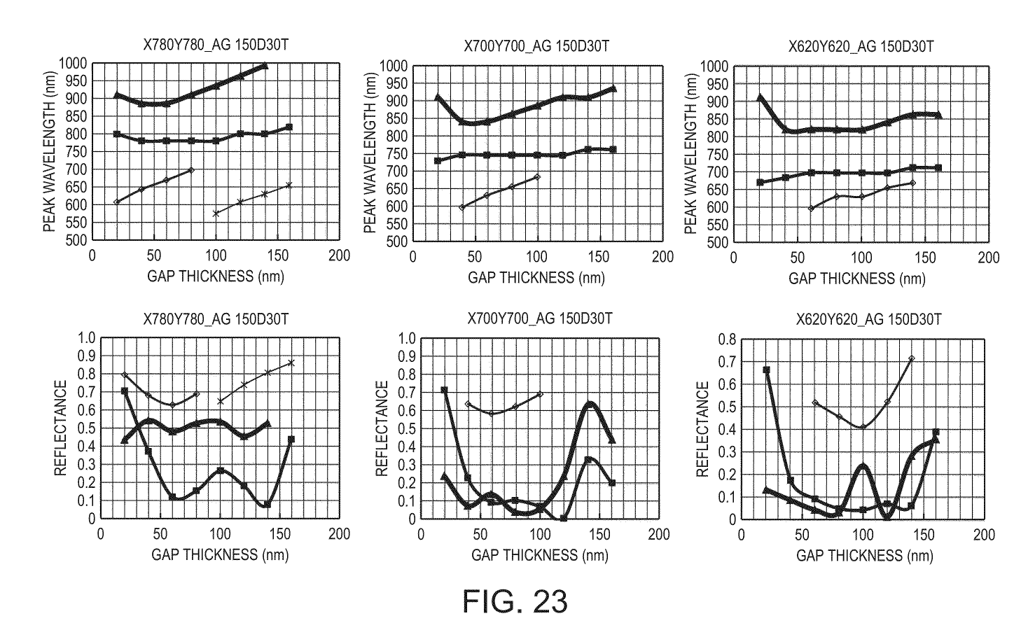

In addition, in Experimental Examples 1 to 7 described above, a model of X600Y600 for the excitation of 633 nm and a model of X780Y780 for the excitation of 785 nm are used, but the model is not limited thereto. FIG. 23 illustrates dependent properties of a wavelength having a peak in a reflectance spectrum of each model of 150D30T_AG and a minimum value of the peak in the reflectance spectrum with respect to the thickness G of the light-transmissive layer in X780Y780, X700Y700, and X620Y620. The diameter D of the metallic particles in this model is 150D by selecting a value at which the enhancement degree increases most.

From FIG. 23, it is found that both of a peak in the vicinity of 780 nm appearing in G=40 nm of X780Y780 (a pitch of 780 nm) and a peak in the vicinity of 880 nm appearing in G=40 nm of X780Y780 (a pitch of 780 nm) are shifted to the short wavelength side by narrowing the pitch. In addition, it is found that reflectance of the peak in the vicinity of 880 nm appearing in G=40 nm of X780Y780 (the pitch of 780 nm) is decreased (an enhancement degree is improved) by narrowing the pitch.

Therefore, it is found that even when the pitch is narrowed to 780 nm, 700 nm, and 620 nm, and the hot spot density (HSD) increases, it is possible to enhance light with an extremely high enhancement degree by setting the range of the thickness G of the light-transmissive layer to the range described in “1.2. Light-Transmissive Layer”.

Specifically, in X780Y780_{—}150D30T_AG_{—}60G, SQRT is 184 at the peak in the vicinity of 790 nm and SQRT is 93 at the peak in the vicinity of 890 nm, and in X620Y620_{—}150D30T_AG_{—}80G, SQRT is 123 at the peak in the vicinity of 710 nm and SQRT is 160 at the peak in the vicinity of 830 nm.

When comparing the intensities of SERS in an ideal state where a peak of the enhancement degree spectrum exists in each wavelength of the excitation light and the scattering light, 184²·93²/(780·780)=481 in X780Y780_{—}150D30T_AG_{—}60G, and 123²·160²/(620·620)=1008 in X620Y620_{—}150D30T_AG_{—}80G, and thus two times or more SERS intensity is obtained by changing the pitch from 780 nm to 620 nm.

Further, for example, it is confirmed that as the model for the excitation of 633 nm, the pitch in the X direction and the Y direction is narrowed, and the same effect as that of X500Y500 in which density of the arrangement of the metallic particles increases is obtained. It is found that the enhancement degree of each peak decreases compared to the model described in the experimental example described above, and SERS intensity is proportionate to Eᵢ²·Eₛ²·HSD, and thus an SERS effect is not greatly decreased by an increase in HSD.

In addition, in all of the experimental examples described above, the shape of the metallic particles is a cylinder, but may be an ellipse or a prism. Further, as the wavelength of the excitation light, HeNe laser of 633 nm and semiconductor laser of 785 nm are considered, but the wavelength is not limited thereto. Further, as the size of the metallic particles, a diameter of 80 nm to 160 nm and a thickness of 30 nm are calculated, but the size is not limited thereto. Furthermore, when the diameter decreases and the thickness decreases or when the diameter increases and the thickness increases, it is possible to obtain a wavelength characteristic identical to or similar to that of each experimental example.

### 4.9. Reference Example

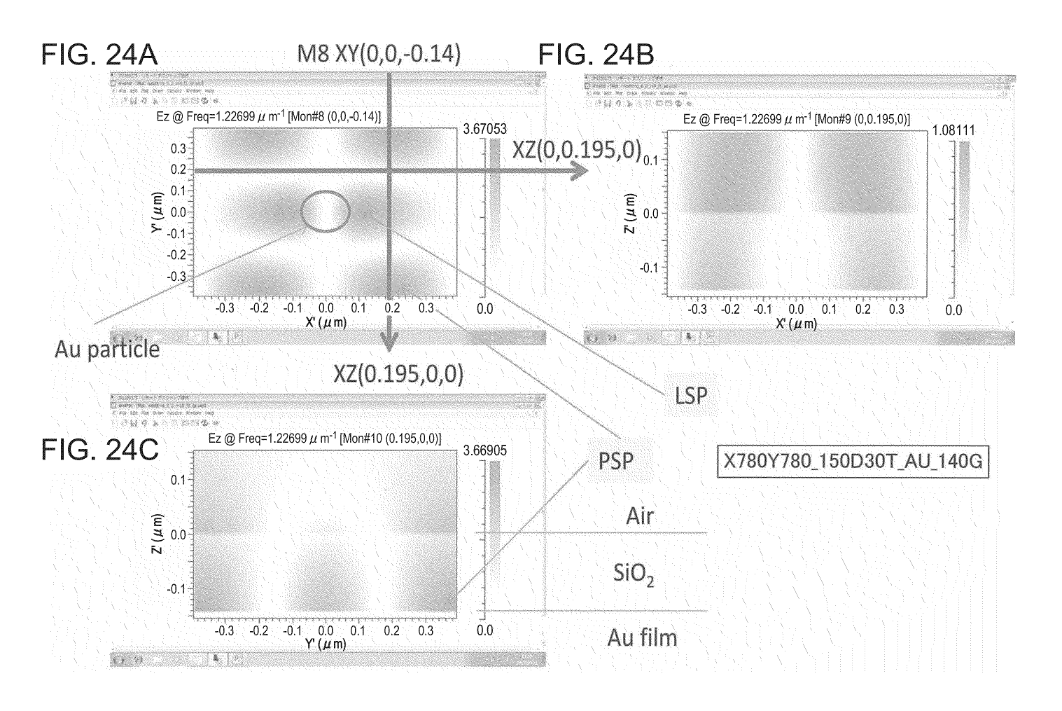

FIGS. 24A to 24C are diagrams illustrating an intensity distribution of E_{z} in XZ (an X pitch/4, 0, 0) of the model of X780Y780_{—}150D30T_AU_{—}140G (the material of the metallic layer is gold, and the material of the light-transmissive layer is SiO₂). FIG. 24A perspectively illustrates the intensity distribution of plasmon in a plan view, and FIGS. 24B and 24C each illustrate the intensity distribution of plasmon in a cross-sectional view of a line illustrated by an arrow in FIG. 24A.

From FIGS. 24A to 24C, the excitation light is linearly polarized light in the X direction, strong LSP is generated in both ends of the metallic particles in the X direction, and PSP is generated in a position between the adjacent metallic particles in a lower portion of LSP described above and in the X direction.

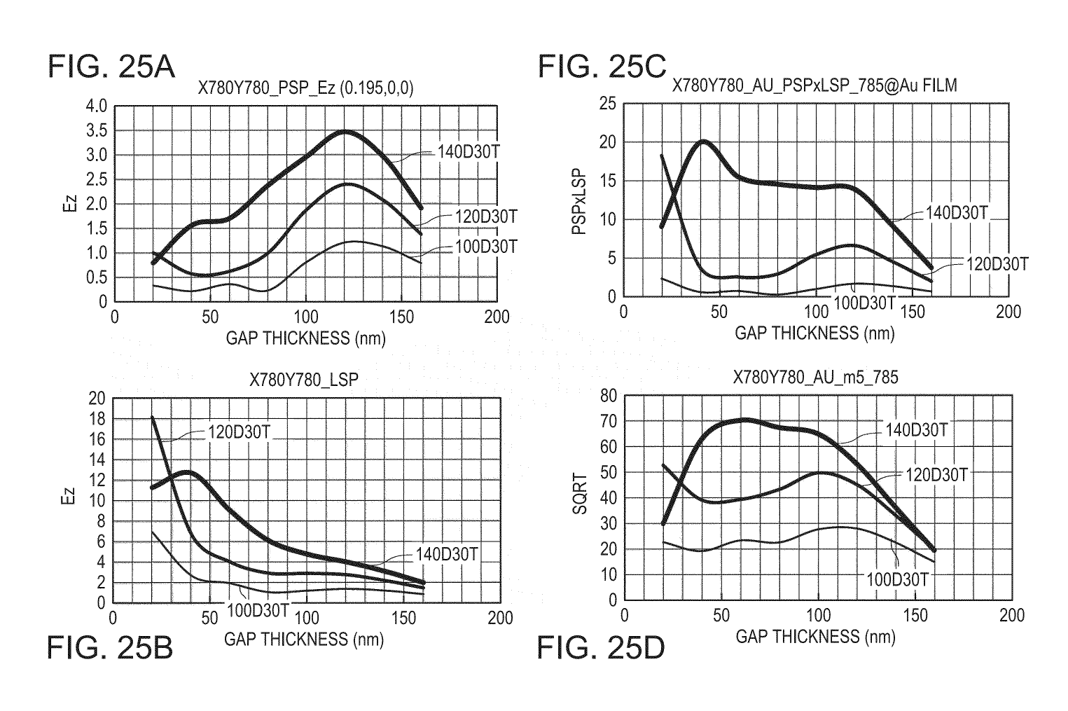

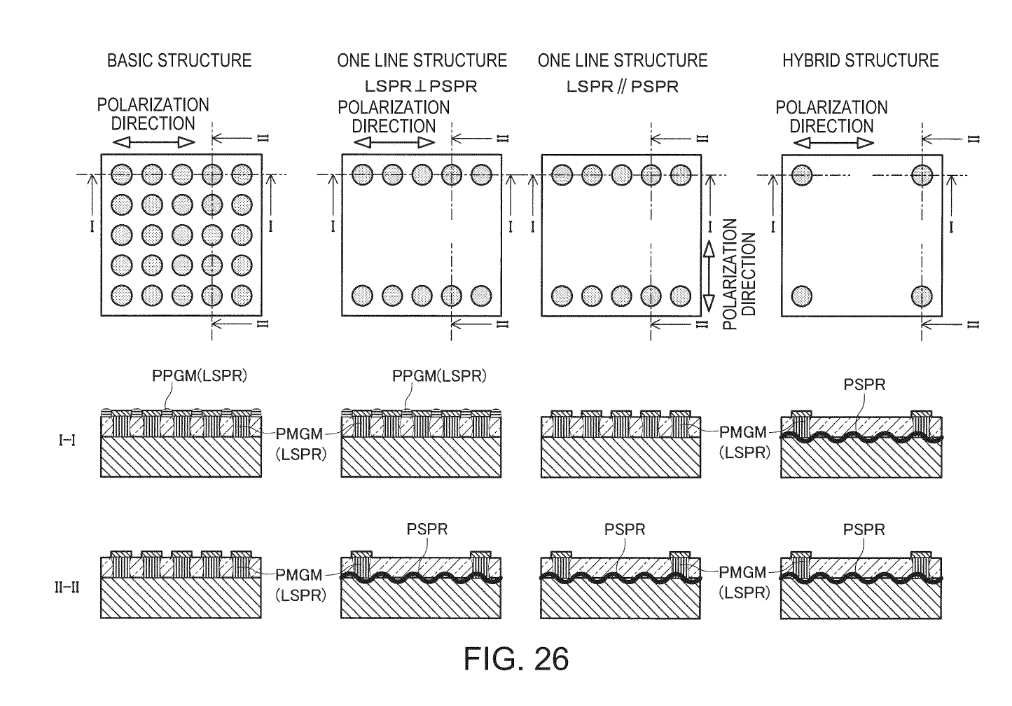

FIGS. 25A to 25D are diagrams for comparing a product of the intensity of PSP and the intensity of LSP when the diameter D of the metallic particles in the model of X780Y780_AU is changed and SQRT. FIG. 25A is dependent properties of PSP with respect to the thickness G of the light-transmissive layer, FIG. 25B is dependent properties of LSP with respect to the thickness G of the light-transmissive layer, FIG. 25C is dependent properties of PSP*LSP (a product of PSP and LSP) with respect to the thickness G of the light-transmissive layer, and FIG. 25D is dependent properties of actually measured SQRT with respect to the thickness G of the light-transmissive layer. From FIGS. 25A to 25D, it is found that the dependent properties of the product of the intensity of PSP and the intensity of LSP with respect to the thickness G of the light-transmissive layer have a trend preferably coincident with that of the dependent properties of SQRT with respect to the thickness G of the light-transmissive layer.

### 5. OTHER MATTERS

FIG. 26 is a schematic view illustrating a relationship between the arrangement of the metallic particles and LSP (Localized Surface Plasmon Resonance (LSPR)) and PSP (Propagating Surface Plasmon Resonance (PSPR)). Herein, for the convenience of the description, a case where LSP is simply generated in the vicinity of the metallic particles has been described. LSP and PSP are electromagnetically and mutually interacted with each other, and thus SPR used in the electric field enhancing element according to the invention is generated.

Here, it is found that in LSP which is able to be generated in the vicinity of the metallic particles, two modes of a mode in which LSP is generated between the adjacent metallic particles (hereinafter, referred to as “Particle-Particle Gap Mode (PPGM)”), and a mode in which LSP is generated between the metallic particles and the metallic layer (having a function of a mirror) (hereinafter, referred to as “Particle-Mirror Gap Mode (PMGM)”) exist (refer to FIG. 26).

The excitation light is incident on the electric field enhancing element, and thus LSP in both of the two modes of PPGM and PMGM is generated. Among them, intensity of LSP in PPGM increases as the metallic particles become closer to each other (a distance between the metallic particles becomes smaller). In addition, intensity of LSP in PPGM increases as an amount of a component (a polarization component) of a vibration in an electric field of the excitation light becomes larger in a parallel direction of the metallic particles which are closer to each other. On the other hand, LSP in the mode of PMGM is not greatly influenced by the arrangement of the metallic particles or the polarization direction of the excitation light, and is generated between the metallic particles and the metallic layer (in a lower portion of the metallic particles) due to the irradiation of the excitation light. Then, as described above, PSP is the plasmon which is transmitted through the surface boundary between the metallic layer and the light-transmissive layer, the excitation light is incident on the metallic layer, and thus PSP is isotropically transmitted through the surface boundary between the metallic layer and the light-transmissive layer.

In FIG. 26, a comparison between a hybrid structure described in the experimental example or the like, and other structures (a basic structure and a one line structure) is schematically illustrated. The polarization direction of the excitation light is illustrated by an arrow in the drawings. Furthermore, herein, the expression of the basic structure, the one line structure, and the hybrid structure is a coined word used for discriminating these structures, and hereinafter, the meaning thereof will be described.

First, the basic structure is a structure in which the metallic particles are densely arranged on the light-transmissive layer, and LSPR in PPGM and LSPR in PMGM are excited due to the irradiation of the excitation light. In this example, LSPR in PPGM is generated in both ends of the metallic particles in the polarization direction of the excitation light, but the basic structure has small anisotropy of the arrangement of the metallic particles, and thus even when the excitation light is not polarized light, similarly, LSPR is generated according to a component of an electric field vector of the excitation light. In the basic structure, as a result of densely arranging the metallic particles, it is difficult for the excitation light to reach the metallic layer, and thus PSPR is rarely generated or is not generated at all, and in the drawings, a schematic broken line indicating PSPR is omitted.

Next, the one line structure is a structure in which the metallic particles are arranged on the light-transmissive layer with intermediate density between the basic structure and the hybrid structure. In the one line structure, there is anisotropy in the arrangement of the metallic particles, and thus LSPR which is generated depends on the polarization direction of the excitation light. Among one line structures, when LSPR⊥PSPR is used (that is, when linearly polarized light is incident in a direction along a direction in which an interval between the metallic particles is narrow), LSPR in PPGM and LSPR in PMGM are excited due to the irradiation of the excitation light. Then, the structure is the one line structure, and thus as a result of sparsely arranging the metallic particles, PSPR (a broken line in the drawings) is generated.

In addition, among the one line structures, when LSPR//PSPR is used (that is, when the linearly polarized light is incident in a direction along a direction in which the interval between the metallic particles is wide), LSPR in PMGM is excited due to the irradiation of the excitation light. In this case, the metallic particles are separated from each other in a direction along the polarization direction of the excitation light, and thus LSPR in PPGM is weak compared to a case of the LSPR⊥PSPR, but this is not illustrated in the drawings. Then, the structure is the one line structure, and thus as a result of sparsely arranging the metallic particles, PSPR (a broken line in the drawings) is generated.

Then, the hybrid structure is a structure in which the metallic particles are sparsely arranged on the light-transmissive layer compared to the basic structure, and LSPR in PMGM is excited due to the irradiation of the excitation light. In this example, the metallic particles are separated from each other, and thus LSPR in PPGM is weakly generated compared to the basic structure, but this is not illustrated in the drawings. In the hybrid structure, as a result of sparsely arranging the metallic particles, PSPR (a broken line in the drawings) is generated.

Furthermore, in FIG. 26, a case where the polarized light is incident is described, but in any structure, when excitation light which is not polarized or circularly polarized light is incident, SPR described above is generated according to a component of a vibration direction in an electric field thereof.

Intensity (an electric field enhancement degree) of entire SPR in each structure correlates with a summation (or a product) of SPR generated in each structure. As described above, a contribution degree of PSPR to the intensity of the entire SPR increases in order of the basic structure<the one line structure<the hybrid structure. In addition, a contribution degree of LSPR (PPGM and PMGM) to the intensity of the entire SPR increases in order of the hybrid structure<the one line structure<the basic structure from a viewpoint of the density (HSD) of the metallic particles. Further, when focusing on LSPR in HSD and PPGM, a contribution degree of LSPR in PPGM to the intensity of the entire SPR increases in order of the hybrid structure<the one line//structure<the one line⊥structure<the basic structure.

As described above, the arrangement of the metallic particles in the electric field enhancing element according to the invention belongs to the hybrid structure of P1=P2, or the one line structure of P1<P2.

In the hybrid structure, the intensity of PSPR is the strongest intensity compared to other structures, and a contribution degree of this PSPR with respect to the entire enhancement degree increases most. Then, the intensity of LSPR in PPGM decreases, the density of the metallic particles decreases, LSPR and PSPR in PMGM are mutually interacted with each other (synergistically bonded to each other) to be electromagnetically strong.

On the other hand, the one line⊥structure and the one line//structure are a structure in which LSPR and PSPR with intermediate intensity are mutually interacted (synergistically bonded) to be electromagnetically strong compared to other structures. In addition, in the one line⊥structure, LSPR and PSPR in PPGM with high intensity are mutually interacted to be electromagnetically strong. In addition, in the one line//structure, LSPR and PSPR in PMGM which are generated with the intermediate density (density higher than that of the hybrid structure) are mutually interacted to be electromagnetically strong.

Therefore, in the one line⊥structure and the one line//structure, at least the density of the metallic particles and the contribution ratio of each SPR, and at least a mechanism of enhancing the electric field are different from that of the basic structure in which PSPR is rarely generated, and the hybrid structure in which LSPR in PPGM is rarely generated.

Then, in the electric field enhancing element according to the invention belonging to the hybrid structure or the one line structure, LSPR and PSPR are synergistically and mutually interacted with each other by the mechanism described above, and thus it is possible to obtain an extremely high electric field enhancement degree.

The invention is not limited to the embodiments described above, but is able to be variously changed. For example, the invention includes a configuration which is substantially identical to the configuration described in the embodiment (for example, a configuration including the same function, the same method, and the same result, or a configuration including the same object and the same effect). In addition, the invention includes a configuration in which a portion which is not an essential portion of the configuration described in the embodiment is displaced. In addition, the invention includes a configuration in which a function effect identical to that of the configuration described in the embodiment is obtained or a configuration in which an object identical to that of the configuration described in the embodiment is able to be attained. In addition, the invention includes a configuration in which a known technology is added to the configuration described in the embodiment.

The entire disclosure of Japanese Patent Application No. 2014-027822, filed Feb. 17, 2014 is expressly incorporated by reference herein.

What is claimed is:

1. An analysis apparatus comprising:
an electric field enhancing element including a metallic layer, a light-transmissive layer which is disposed on the metallic layer and transmits excitation light, and a plurality of metallic particles which is disposed on the light-transmissive layer, and is arranged in a first direction and a second direction intersecting with the first direction;
a light source irradiating the electric field enhancing element with at least one of linearly polarized light which is polarized in the first direction, linearly polarized light which is polarized in the second direction, and circularly polarized light as the excitation light; and
a detector detecting light emitted from the electric field enhancing element,
wherein localized surface plasmon excited to the metallic particles and propagating surface plasmon excited to a surface boundary between the metallic layer and the light-transmissive layer are electromagnetically interacted, and
when a thickness of the light-transmissive layer is G [nm], an effective reflective index of the light-transmissive layer is n_{eff}, and a wavelength of the excitation light is λᵢ [nm], a relationship of the following expression (1) is satisfied:
20 [nm]<G·(n_{eff}/1.46)≦140 [nm]·(λᵢ/785 [nm]) (1).

2. An analysis apparatus, comprising:
an electric field enhancing element including a metallic layer, a light-transmissive layer which is disposed on the metallic layer and transmits excitation light, and a plurality of metallic particles which is disposed on the light-transmissive layer, and is arranged in a first direction and a second direction intersecting with the first direction;
a light source irradiating the electric field enhancing element with at least one of linearly polarized light which is polarized in the first direction, linearly polarized light which is polarized in the second direction, and circularly polarized light as the excitation light; and
a detector detecting light emitted from the electric field enhancing element,
wherein localized surface plasmon excited to the metallic particles and propagating surface plasmon excited to a surface boundary between the metallic layer and the light-transmissive layer are electromagnetically interacted,
the light-transmissive layer is formed of a laminated body in which m layers are laminated,
m is a natural number,
the light-transmissive layer is formed by laminating a first light-transmissive layer, a second light-transmissive layer, . . . , a (m−1)-th light-transmissive layer, and a m-th light-transmissive layer in this order from the metallic particle side to the metallic layer side, and
when a refractive index in the vicinity of the metallic particles is n₀, an angle between a normal direction of the metallic layer and an incident direction of the excitation light is θ₀, an angle between the normal direction of the metallic layer and an incident direction of refracting light of the excitation light in the m-th light-transmissive layer with respect to the metallic layer is θₘ, a refractive index of the m-th light-transmissive layer is nₘ, a thickness of the m-th light-transmissive layer is Gₘ [nm], and a wavelength of the excitation light is λᵢ [nm], relationships of the following expression (2) and expression (3) are satisfied:
$$\begin{matrix} {n}_{0} \cdot sin {θ}_{0} = {n}_{m} \cdot sin {θ}_{m} & ( 2 ) \\ 20 [ nm ] < {\sum }_{m = 1}^{m} \{ ( {G}_{m} \cdot cos {θ}_{m} ) \cdot ( {n}_{m} / 1.46 ) \} ≦ 140 [ nm ] \cdot {λ}_{i } / 785 [ nm ] . & ( 3 )\end{matrix}$$

3. The analysis apparatus according to claim 1,
wherein a first pitch P1 at which the metallic particles are arranged in the first direction, and a second pitch P2 at which the metallic particles are arranged in the second direction are identical to each other.

4. The analysis apparatus according to claim 2,
wherein a first pitch P1 at which the metallic particles are arranged in the first direction, and a second pitch P2 at which the metallic particles are arranged in the second direction are identical to each other.

5. An analysis apparatus, comprising:
an electric field enhancing element including a metallic layer, a light-transmissive layer which is disposed on the metallic layer and transmits excitation light, and a plurality of metallic particles which is disposed on the light-transmissive layer, and is arranged in a first direction at a first pitch and arranged in a second direction intersecting with the first direction at a second pitch;
a light source irradiating the electric field enhancing element with at least one of linearly polarized light which is polarized in the first direction, linearly polarized light which is polarized in the second direction, and circularly polarized light as the excitation light; and
a detector detecting light emitted from the electric field enhancing element,
wherein arrangement of the metallic particles of the electric field enhancing element satisfies a relationship of the following expression (4),
P1<P2≦Q+P1 (4)
in which P1 is the first pitch, P2 is the second pitch, and Q is a pitch of a diffraction grating satisfying the following expression (5) when an angular frequency of localized plasmon excited to a row of the metallic particles is ω, a dielectric constant of metal configuring the metallic layer is ∈ (ω), a dielectric constant in the vicinity of the metallic particles is ∈, a speed of light in vacuum is c, and an inclined angle from a thickness direction of the metallic layer which is an irradiation angle of the excitation light is θ,
(ω/c)·{∈·∈(ω)/(∈+∈(ω))}^{1/2}=∈^{1/2}·(ω/c)·sin θ+2aπ/Q(a=±1,±2, . . . ) (5), and
when a thickness of the light-transmissive layer is G [nm], an effective reflective index of the light-transmissive layer is n_{eff}, and a wavelength of the excitation light is λᵢ [nm], a relationship of the following expression (1) is satisfied:
20 [nm]<G·(n_{eff}/1.46)≦140 [nm]·(λᵢ/785 [nm]) (1).

6. The analysis apparatus according to claim 1,
wherein the first pitch P1 satisfies a relationship of 60 [nm]≦P1≦1310 [nm].

7. The analysis apparatus according to claim 2,
wherein the first pitch P1 satisfies a relationship of 60 [nm]≦P1≦1310 [nm].

8. The analysis apparatus according to claim 4,
wherein the first pitch P1 satisfies a relationship of 60 [nm]≦P1≦1310 [nm].

9. The analysis apparatus according to claim 1,
wherein the second pitch P2 satisfies a relationship of 60 [nm]≦P2≦1310 [nm].

10. The analysis apparatus according to claim 2,
wherein the second pitch P2 satisfies a relationship of 60 [nm]≦P2≦1310 [nm].

11. The analysis apparatus according to claim 4,
wherein the second pitch P2 satisfies a relationship of 60 [nm]≦P2≦1310 [nm].

12. The analysis apparatus according to claim 1,
wherein the light-transmissive layer includes a layer selected from silicon oxide, titanium oxide, aluminum oxide, silicon nitride, and tantalum oxide.

13. The analysis apparatus according to claim 2,
wherein the light-transmissive layer includes a layer selected from silicon oxide, titanium oxide, aluminum oxide, silicon nitride, and tantalum oxide.

14. The analysis apparatus according to claim 4,
wherein the light-transmissive layer includes a layer selected from silicon oxide, titanium oxide, aluminum oxide, silicon nitride, and tantalum oxide.

15. The analysis apparatus according to claim 1,
wherein the metallic layer includes a layer formed of gold, silver, copper, platinum, or aluminum.

16. The analysis apparatus according to claim 1,
wherein a ratio of intensity of localized surface plasmon excited to a corner portion of the metallic particles on a side away from the light-transmissive layer to intensity of localized surface plasmon excited to a corner portion of the metallic particles on a side close to the light-transmissive layer is constant regardless of the thickness of the light-transmissive layer.

17. An electronic device, comprising:
the analysis apparatus according to claim 1;
a calculation unit which calculates medical health information on the basis of detection information from the detector;
a storage unit which stores the medical health information; and
a display unit which displays the medical health information.

18. An electronic device, comprising:
the analysis apparatus according to claim 2;
a calculation unit which calculates medical health information on the basis of detection information from the detector;
a storage unit which stores the medical health information; and
a display unit which displays the medical health information.

19. An electronic device, comprising:
the analysis apparatus according to claim 4;
a calculation unit which calculates medical health information on the basis of detection information from the detector;
a storage unit which stores the medical health information; and
a display unit which displays the medical health information.
